(19) United States
(12) Patent Application Publication
(10) Pub. No.: US 2005/0014780 A1
(43) Pub. Date: Jan. 20, 2005

(54) Process for preparing 5-sulfonamido-8-hydroxy-1,6-naphthyridine-7-carboxamides

(76) Inventors: Peter E. Maligres, Fanwood, NJ (US); David Askin, Warren, NJ (US)

Correspondence Address: MERCK AND CO INC, P O BOX 2000, RAHWAY, NJ, 070650907, US

(21) Appl. No.: 10/486,535

(22) PCT Filed: Aug. 13, 2002
(86) PCT No.: PCT/US02/27151

Related U.S. Application Data
(60) Provisional application No. 60/313,376, filed on Aug. 17, 2001.

(51) Int. Cl. C07D 471/02; A61K 31/4745

(52) U.S. Cl. 514/300; 546/123

(57) ABSTRACT

The preparation of 5-sulfonamido-8-hydroxy-1,6-naphthyridine-7-carboxamides is disclosed. A 5-halo-8-hydroxy-1,6-napthyridine-7-carboxylic acid or acid ester in which the hydroxy is derivatized with a protecting group is reacted with a sulfonamide (e.g., an alkanesulfonamide, N-alkyl alkanesulfonamide, or alkanesultam) in the presence of a copper promoter and a chelating agent, followed by deprotection of the hydroxy group, and then coupling with an amine to obtain the 5-sulfonamido-8-hydroxy-1,6-naphthyridine-7-carboxamide. Alternatively, the hydroxy-protected 5-halo-8-hydroxy-1,6-napthyridine-7-carboxylic acid (or ester) is first coupled with an amine, the resulting carboxamide reacted with a sulfonamide followed by deprotection of the hydroxy group to obtain the 5-sulfonamido-8-hydroxy-1,6-naphthyridine-7-carboxamide. The 5-sulfonamido-8-hydroxy-1,6-naphthyridine-7-carboxamides are inhibitors of HIV integrase and are useful for treating HIV infection, preventing HIV infection, delaying the onset of AIDS, and treating AIDS.

### FIELD OF THE INVENTION

The present invention is directed to the preparation of 5-sulfonamido-8-hydroxy-1,6-naphthyridine-7-carboxamides via processes which involve the condensation of sulfonamides (e.g., alkanesulfonamides, N-alkyl alkanesulfonamides, or alkanesultams) with suitable halonaphthyridine intermediates in the presence of a copper promoter and copper chelating agent. The 5-sulfonamido-8-hydroxy-1,6-naphthyridine-7-carboxamides are HIV integrase inhibitors useful for preventing HIV infection, treating HIV infection, delaying the onset of AIDS, and treating AIDS.

### BACKGROUND OF THE INVENTION

The HIV retrovirus is the causative agent for AIDS. The HIV-1 retrovirus primarily uses the CD4 receptor (a 58 kDa transmembrane protein) to gain entry into cells, through high-affinity interactions between the viral envelope glycoprotein (gp 120) and a specific region of the CD4 molecule found in T-lymphocytes and CD4 (+) T-helper cells (Lasky L. A. et al., Cell 1987, 50: 975-985). HIV infection is characterized by an asymptomatic period immediately following infection that is devoid of clinical manifestations in the patient. Progressive HIV-induced destruction of the immune system then leads to increased susceptibility to opportunistic infections, which eventually produces a syndrome called ARC (AIDS-related complex) characterized by symptoms such as persistent generalized lymphadenopathy, fever, and weight loss, followed itself by full blown AIDS.

After entry of the retrovirus into a cell, viral RNA is converted into DNA, which is then integrated into the host cell DNA. Integration of viral DNA is an essential step in the viral life cycle. Integration is believed to be mediated by integrase, a 32 kDa enzyme, in three steps: assembly of a stable nucleoprotein complex with viral DNA sequences; cleavage of two nucleotides from the 3′ termini of the linear proviral DNA; and covalent joining of the recessed 3′ OH termini of the proviral DNA at a staggered cut made at the host target site. The fourth step in the process, repair synthesis of the resultant gap, may be accomplished by cellular enzymes.

Certain 5-sulfonamido-8-hydroxy-1,6-naphthyridine-7-carboxamides constitute a class of inhibitors of HIV integrase and HIV replication. Compounds of this class include, but are not limited to, compounds of Formula (AA): 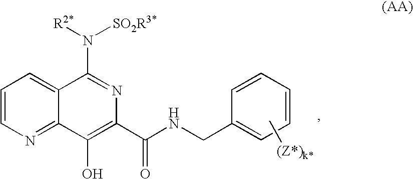
wherein:
R²*is H or alkyl;
R³*is alkyl;
or R²*and R³*together with the —NSO₂— moiety to which they are attached form a sultam group of formula 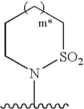
   wherein m*=0, 1 or 2;
each Z*is independently H or a substituent such as halogen, alkyl, haloalkyl, or alkoxy; and
k*is an integer from zero to 5.

Exemplary of the compounds of Formula (AA) is: 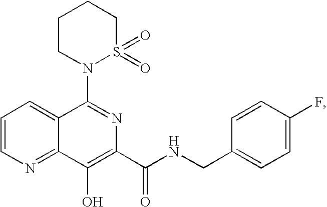
which is alternatively referred to herein as Compound 15.

This class of HIV integrase inhibitors can be prepared by routes involving the condensation of a sulfonamide such as an N-alkyl alkanesulfonamide or an alkanesultam with a suitable 5-halo-8-hydroxy-1,6-naphthyridine intermediate. The route for preparing compounds of Formula (AA) is representative and is shown in Scheme A as follows.

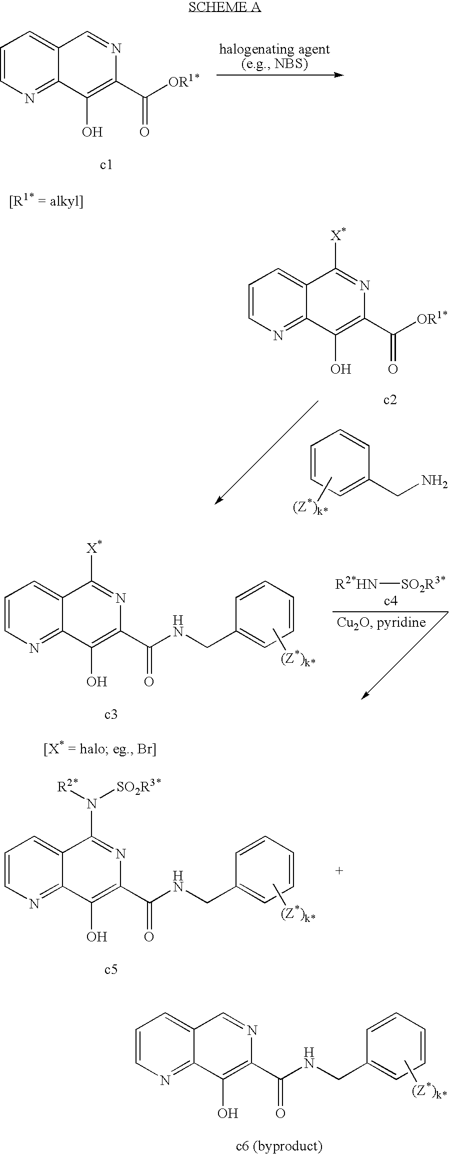

The preparation includes halogenation of an alkyl 8-hydroxy-naphthyridine carboxylate (c1) with a halogenation agent such as N-bromosuccinimide, coupling the halogenated ester (c2) with substituted or unsubstituted benzylamine, and then condensing the 5-halo-8-hydroxy-naphthyridine carboxamide (c3) with a sulfonamide (c4) at elevated temperature (e.g., about 120° C.) in the presence of a copper promoter (e.g., copper(I) oxide) to afford the desired sulfonamidonaphthyridine product (c5). The yield of this step is relatively low (e.g., typically about 40% or less in the preparation of Compound 15) with the production of a significant amount of naphthyridine carboxamide c6 as byproduct. There are also tar-like byproducts which are difficult to remove from the desired product (e.g., cannot be separated by filtration). In addition it can be difficult to separate the copper from the desired product c5. Further description of this route can be found in WO 02/30930 (see, e.g., Scheme 17).

There is a need for alternative and/or improved processes for preparing these integrase inhibitors which can provide a higher yields and/or can facilitate the workup of the intermediate and/or final products.

References of interest with respect to the present invention include the following:
Coutts et al., J. Chem. Soc. Perkin I 1975, 2445-2446 discloses the reaction of certain N-arylsulfonamides with certain aryl bromides in the presence of copper powder and potassium carbonate to give the corresponding N,N-diarylsulfonamides.
Lindley, Tetrahedron 1984, 40: 1433-1456 is a review of the copper-assisted nucleophilic substitution of aryl halogen and includes a discussion of the Ullman condensation of certain aryl halides with ammonia, amines, imides and amides.
Kandzia et al., Tetrahedron: Asymmetry 1993, 4: 39-42 discloses the preparation of camphor sultam-based chiral bipyridines and phenanthrolines by reacting the camphor sultam with the bipyridine or phenanthroline in refluxing collidine in the presence of Cu(I) oxide.
Chemical Abstracts No. 122:314455g, 1995 (an abstract of JP 06135934) discloses heating 2,6-dichloro-3-(trifluoromethyl)pyridine and ethanesulfonyl amide in DMSO containing Na2CO3 to give a 2:1 mixture of N-(6-chloro-3-(trifluoromethyl)-2-pyridinyl)ethanesulfonamide and N-(6-chloro-5-(trifiuoromethyl)-2-pyridinyl)ethanesulfonamide.

### SUMMARY OF THE INVENTION

The present invention is directed to a process for preparing 5-sulfonamido-8-hydroxy-1,6-naphthyridine-7-carboxamide compounds, which are HIV integrase inhibitors useful for treating HIV infection, preventing HIV infection, treating AIDS, and delaying the onset of AIDS. The present invention includes a process for preparing a compound of Formula (VIII): 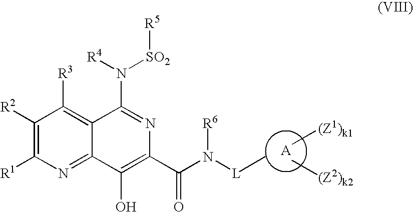
which comprises:
(C) reacting a compound of Formula (IIIa) or (IIIb): 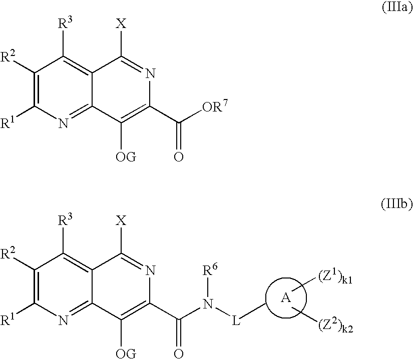
   with a sulfonamide of Formula (IV):
   R⁵SO₂—NHR⁴ (IV)
   in solvent and in the presence of a copper promoter and a copper chelating agent to obtain a compound of Formula (Va) or (Vb): 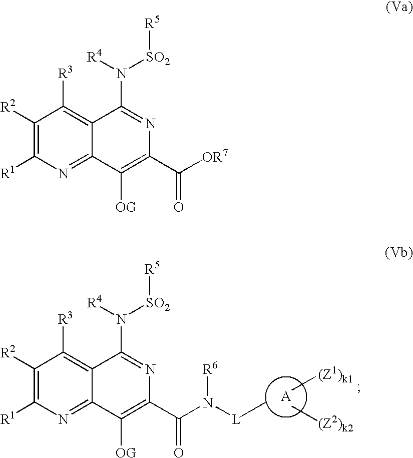
(D) when the compound resulting from Step C is Compound Va,
   (D1) treating Compound Va with a phenol deprotecting agent to obtain a compound of Formula (VI): 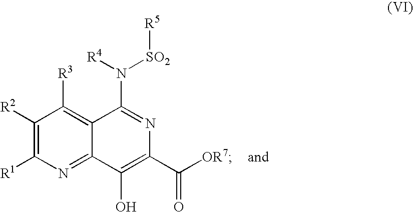
   (D2) coupling Compound VI with an amine of Formula (VII): 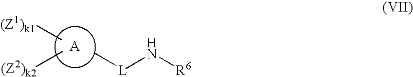
to obtain Compound VIII; and
(E) when the compound resulting from Step C is Compound Vb, treating Compound Vb with a phenol deprotecting agent to obtain Compound VIII;
   wherein
A is phenyl or phenyl fused to a carbocycle to form a fused carbocyclic ring system;
G is a phenol protective group; or alternatively and with the proviso that the reactant in Step C is Compound IIIa, G and R⁷ together with the phenolic oxygen moiety and carbonyloxy moiety to which they are attached form a phenol protective cyclic group of formula: 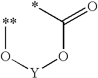
   wherein * and ** respectively denote the points of fusion to ring carbons 7 and 8 in the naphthyridine ring; and Y is —C(R^{c})(R^{d})- or -B(Rₑ)-;
L is a linker connecting a ring atom of A to the nitrogen of the —N(R⁶)-moiety, wherein L is
   (i) a single bond connecting ring system A directly to N(R⁶),
   (ii)-(C₁₋₆ alkyl)-,
   (iii)-(C₂₋₆ alkenyl)-, or
   (iv)-(C₀₋₆ alkyl)-(C₃₋₆ cycloalkyl)-(C₀₋₆ alkyl)-;
X is halo;
each Z¹ is a substituent on A independently selected from the group consisting of:
   (1) —H,
   (2) —C₁₋₆ alkyl, which is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂,
   (3) —O—C₁₋₆ alkyl, which is optionally substituted with from 1 to 7 substituents each of which is independently halogen, —O—C₁₋₆ alkyl, —OH, or —SR^{a},
   (4) —OH,
   (5) halogen,
   (6) —NO₂,
   (7) —CN,
   (8) —C(═O)R^{a},
   (9) —CO₂R^{a},
   (10) —SR^{a},
   (11) —N(R^{b})₂,
   (12) —C(═O)N(R^{a})₂,
   (13) —SO₂R^{a},
   (14) —N(R^{a})SO₂R^{a}, and
   (15) —C₂₋₅ alkenyl;
      k1 is an integer equal to zero, 1, 2, 3, 4 or 5;
      each Z² is a substituent on A independently selected from the group consisting of:
   (1) —H,
   (2) aryl,
   (3) —O-aryl,
   (4) —C₁₋₆ alkyl-aryl,
   (5) —O—C₁₋₆ alkyl-aryl,
   (6) heteroaryl
   (7)—O-heteroaryl,
   (8)-C₁₋₆ alkyl-heteroaryl, and
   (9) —O—C₁₋₆ alkyl-heteroaryl,
      wherein the aryl in any of (2) to (5) or the heteroaryl in any of (6) to (9) is optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
         k₂ is an integer equal to zero, 1, or 2;
         each of R¹, R² and R³ is independently:
   (1) —H,
   (2) —C₁₋₆ alkyl, which is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂,
   (3) —O—C₁₋₆ alkyl, which is optionally substituted with from 1 to 7 substituents each of which is independently halogen, —O—C₁₋₆ alkyl, —OH, or —SR^{a},
   (4) —OH,
   (5) halogen,
   (6) —NO₂,
   (7) —CN,
   (8) —C(═O)R^{a},
   (9) —CO₂R^{a},
   (10) —SR^{a},
   (11) —N(R^{b})₂,
   (12) —C(═O)N(R^{a})₂,
   (13) —SO₂R^{a},
   (14) —N(R^{a})SO₂R^{a}, and
   (15) —C₂₋₅ alkenyl;
   (16) aryl,
   (17) —O-aryl,
   (18) —C₁₋₆ alkyl-aryl,
   (19) —O—C₁₋₆ alkyl-aryl,
   (20) heteroaryl (21)—O-heteroaryl,
   (22) —C₁₋₆ alkyl-heteroaryl, and
   (23) —O—C₁₋₆ alkyl-heteroaryl,
      wherein the aryl in any of (16) to (19) or the heteroaryl in any of (20) to (23) is optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁴ is —H, —C₁₋₆ alkyl, or aryl; wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁵ is C₁₋₆ alkyl or aryl; wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂; or alternatively R⁴ and R⁵ together with the —NSO₂— moiety to which they are attached form a sultam group of formula: 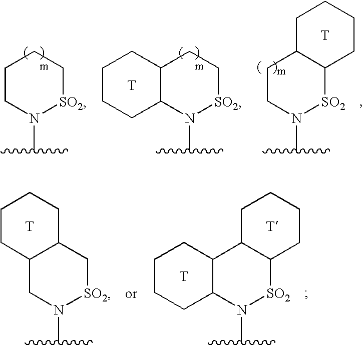
   wherein T and T′ are each independently a 6-membered carbocyclic ring which is saturated, partially unsaturated, or aromatic; m is an integer equal to zero, 1, or 2; and the sultam group is optionally substituted with from 1 to 4 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁶ is —H or —C₁₋₆ alkyl, wherein the alkyl is optionally substituted with from 1 to 7 substituents independently selected from halogen, —O—C₁₋₆ alkyl, —O—C₁₋₆ haloalkyl, —N(R^{b})₂, and —CO₂R^{a};
R⁷ is —H, —C₁₋₆ alkyl or aryl, wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, —C(═O)N(R^{a})₂, or phenyl; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
each R^{a} is independently —H or —C₁₋₆ alkyl;
each R^{b} is independently —C₁₋₆ alkyl; and
R^{c} and R^{d} are each independently —H or —C₁₋₆ alkyl which is optionally substituted with from 1 to 7 substituents each of which is independently halogen, —O—C₁₋₆ alkyl, or —O—C₁₋₆ haloalkyl;
R^{e} is —C₁₋₆ alkyl, —O—C₁₋₆ alkyl, aryl, or —O-aryl; wherein the alkyl is optionally substituted with from 1 to 7 substituents each of which is independently halogen, —O—C₁₋₆ alkyl, or —O—C₁₋₆ haloalkyl; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —CHO, —C(═O)—C₁₋₆ alkyl, —CO₂H, —CO₂—C₁₋₆ alkyl, —SH, —S—C₁₋₆ alkyl, —N(—C₁₋₆ alkyl)₂, —C(═O)NH₂, or —C(═O)N(—C₁₋₆ alkyl)₂;
   and wherein each aryl is independently phenyl, naphthyl, anthryl, or phenanthryl;
   and each heteroaryl is independently a 5- or 6-membered heteroaromatic ring containing from 1 to 3 heteroatoms selected from N, O and S and a balance of carbon atoms.

The process of the present invention is distinguished from the previous process by the use of a protected hydroxynaphthyridine reactant in the Ullman-type copper-promoted condensation with the sulfonamide (Step C above). The process of the present invention is further distinguished from the previous process by its use of a copper-chelating agent in the copper-promoted condensation. The condensation reaction of Step C of the present invention has been found to proceed cleanly with little or no competing overreduction (i.e., little or no formation of byproducts analogous to c6), resulting in substantially improved yields of sulfonamide product compared to the previous process. The use of the protected hydroxynaphthyridine reactant in the Ullman-type condensation reaction of the present invention has also been found to produce far fewer tar-like byproducts than the previous process, which facilitates the workup of the sulfonamide product. In addition, the copper is typically much easier to separate from the sulfonamide product by washing. While not wishing to be bound by any theory, it is believed that the copper does not complex to the derivatized hydroxy group —OG (in Compound IIIa or IIIb) as strongly as to the free —OH group (in Compound c3).

Other embodiments, aspects and features of the present invention are either further described in or will be apparent from the ensuing description, examples and appended claims.

### DETAILED DESCRIPTION OF THE INVENTION

The present invention includes the preparation of naphthyridine carboxamides of Formula (VIII) by the process set forth above in the Summary of the Invention. The naphthyridine carboxamides of Formula (VIII) are inhibitors of HIV integrase. Representative compounds embraced by Formula (VI) have been tested in an integrase inhibition assay in which strand transfer is catalyzed by recombinant integrase, and have exhibited IC₅₀'s of less than about 100 micromolar. The strand transfer assay is described in Example 193 of WO 02/30930. Representative compounds have also been tested in an assay for the inhibition of acute HIV infection of T-lymphoid cells conducted in accordance with Vacca et al., Proc. Natl. Acad. Sci. USA 1994, 91: 4096-4100, and have exhibited IC₉₅'s of less than about 20 micromolar.

In one embodiment of the process of the invention, A in the compounds of Formula IIIb, Vb, VII, and VIII is

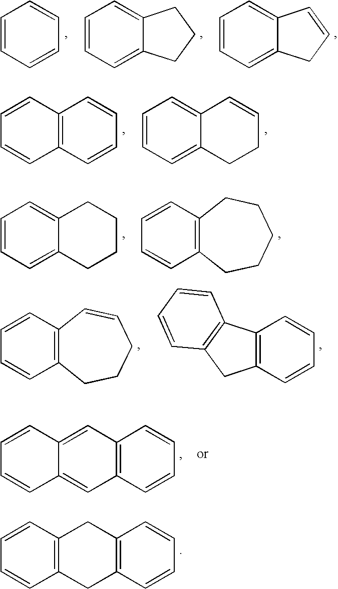

In another embodiment of the process of the present invention, A is

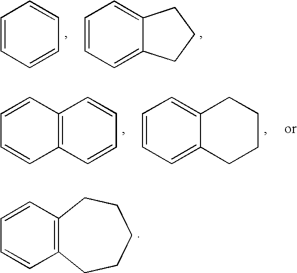

In still another embodiment of the process, A is

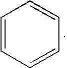

Other embodiments of the process of the invention include the process as originally defined above or as defined in any one of the preceding embodiments, wherein the —OG group in Compounds IIIa and Va or Compounds IIIb and Vb is an ether, a silyl ether, a carboxylic ester, a carbonate, a phosphinate or a sulfonate. In an aspect of each of these embodiments, the —OG group in Compounds IIIa and Va or IIIb and Vb is an ether, a silyl ether, a carboxylic ester, or a sulfonate.

Still other embodiments of the process of the invention include the process as originally defined or as defined in any one of the preceding embodiments, wherein G is:
(1) —C₁₋₆ alkyl,
(2)-C₁₋₆ alkyl-aryl,
(3)-C₁₋₆ alkyl-O-C₁₋₆ alkyl,
(4) —C₁₋₆ alkyl-O-C₁₋₆ alkyl-aryl,
(5) —C₃₋₈ cycloalkyl,
(6) allyl,
(7)-Si(C₁₋₆ alkyl)ₙ(aryl)₃₋ₙ, wherein n is an integer equal to zero, 1, 2, or 3;
(8) —C(═O)—C₁₋₆ alkyl,
(9) —C(═O)-aryl,
(10) —C(═O)-C₁₋₆ alkyl-aryl,
(11) —C(═O)—O—C₁₋₆ alkyl,
(12) —C(═O)—O-aryl,
(13) —C(═O)—O—C₁₋₆ alkyl-aryl,
(14) —SO₂—C₁₋₆ alkyl,
(15) —SO₂—C₁₋₆ haloalkyl,
(16) —SO₂-aryl, or
(17) —P(O)(—C₁₋₆ alkyl)₂
   wherein the aryl in (2), (4), (9), (10), (12), (13), or (16), and each aryl in (7) is optionally substituted with from 1 to 5 substituents each of which is independently halogen, —C₁₋₄ alkyl, —O—C₁₋₄ alkyl, or nitro.

In an aspect of each of the preceding embodiments defined in the preceding paragraph, G is —SO₂-C₁₋₆ alkyl, —SO₂—C₁₋₆ haloalkyl, or —SO₂-aryl, wherein the aryl is optionally substituted with from 1 to 5 substituents each of which is independently halogen, —C₁₋₄ alkyl, —O-C₁₋₄ alkyl, or nitro. In a feature of this aspect, G is CH₃SO₂—, CF₃SO₂—, or p-toluenesulfonyl. In another feature of this aspect, G is p-toluenesulfonyl.

Other embodiments of the process of the invention include the process as originally defined above or as defined in any one of the preceding embodiments, wherein X is chloro or bromo; or is bromo; or is chloro.

Other embodiments of the process of the invention include the process as originally defined above or as defined in any one of the preceding embodiments, wherein L is —(C₁₋₆ alkyl)-. In an aspect of each of these embodiments, L is —(CH₂)₁₋₄-. In another aspect of each of these embodiments, L is —CH₂—.

Other embodiments of the process of the invention include the process as originally defined above or as defined in any one of the preceding embodiments, wherein each Z¹ is independently selected from the group consisting of:
(1) —H,
(2)-C₁₋₄ alkyl, which is optionally substituted with from 1 to 5 substituents each of which is independently halogen, —O—C₁₋₄ alkyl, or —O-C₁₋₄ haloalkyl,
(3) —O—C₁₋₄ alkyl, which is optionally substituted with from 1 to 5 substituents each of which is independently halogen or —O-C₁₋₄ alkyl,
(4) halogen,
(5) —CN,
(6) —C(═O)R^{a},
(7) —CO₂R^{a},
(8) —SR^{a},
(9) —N(R^{b})₂,
(10) —C(═O)N(R^{a})₂,
(11) —SO₂R^{a},
(12) —N(R^{a})SO₂R^{a}, and
(13) —C₂₋₅ alkenyl.

In an aspect of each of the embodiments of the process set forth in the preceding paragraph, each Z¹ is independently —H, —C₁₋₄ alkyl, —(CH₂)₀₋₂CF₃, —O—C₁₋₄ alkyl, —O—(CH₂)₀₋₂CF₃, or halo selected from —F, —Cl and —Br.

Other embodiments of the process of the invention include the process as originally defined above or as defined in any one of the preceding embodiments, wherein each Z² is independently selected from the group consisting of:
(1) —H,
(2) aryl,
(3) —O-aryl,
(4)-C₁₋₄ alkyl-aryl, and
(5) —O-C₁₋₄ alkyl-aryl,
   wherein the aryl in any of (2) to (5) is phenyl or naphthyl and is optionally substituted with from 1 to 5 substituents each of which is independently halogen, —C₁₋₄ alkyl, —C₁₋₄ haloalkyl, —O-C₁₋₄ alkyl, or —O-C₁₋₄ haloalkyl.

Other embodiments of the process of the invention include the process as originally defined above or as defined in any one of the preceding embodiments, wherein k1 is an integer equal to zero, 1, 2 or 3; or equal to zero, 1 or 2; or equal to zero or 1; or equal to zero; or equal to 1; or equal to 2; or equal to 3.

Other embodiments of the process of the invention include the process as originally defined above or as defined in any one of the preceding embodiments, wherein k2 is an integer equal to zero or 1; or equal to zero; or equal to 1; or equal to 2.

Other embodiments of the process of the invention include the process as originally defined above or as defined in any of the preceding embodiments, wherein k1 is an integer equal to zero, 1, 2 or 3; and k2 is an integer equal to zero or 1.

Still other embodiments of the process of the invention include the process as originally defined above or as defined in any of the preceding embodiments, wherein each Z¹ is independently —H, —C₁₋₄ alkyl, —(CH₂)₀₋₂CF₃, —O-C₁₋₄ alkyl, —O—(CH₂)₀₋₂CF₃, or halo selected from —F, —Cl and —Br; k1 is zero, 1 or 2; and k2 is zero.

Other embodiments of the process of the invention include the process as originally defined above or as defined in any of the preceding embodiments, wherein each of R¹, R² and R³ is independently:
(1) —H,
(2) —C₁₋₄ alkyl
(3)—(CH₂)₀₋₂CF₃,
(4) —O-C₁₋₄ alkyl,
(5) —O—(CH₂)₀₋₂CF₃,
(6) halogen selected from —F, —Cl and —Br,
(7) phenyl,
(8) —O-phenyl,
(9) —(CH₂)12-phenyl, or
(10) —O—(CH₂)₁₋₂-phenyl,
   wherein the phenyl in any of (7) to (10) is optionally substituted with from 1 to 4 substituents each of which is independently —F, —Cl, —Br, —C₁₋₄ alkyl, —C₁₋₄haloalkyl, —O-C₁₋₄ alkyl, or —O-C₁₋₄ haloalkyl.

In an aspect of each of the embodiments of the process set forth in the preceding paragraph, R¹ is —H, and R² and R³ are each as heretofore defined. In another aspect of each of these embodiments, each of R¹, R² and R³ is —H.

Other embodiments of the process of the invention include the process as originally defined above or as defined in any of the preceding embodiments, wherein R⁴ and R⁵ in compounds of Formula IV, Va, Vb, VI, and VIII are defined as follows:
R⁴ is —H, —C₁₋₄ alkyl, or phenyl, wherein the alkyl is optionally substituted with from 1 to 5 substituents each of which is independently halogen, —O—C₁₋₄ alkyl, or —O—C₁₋₄ haloalkyl; the phenyl is optionally substituted with from 1 to 4 substituents each of which is independently halogen, —C₁₋₄ alkyl, —C₁₋₄ haloalkyl, —O-C₁₋₄ alkyl, or —O-C₁₋₆ haloalkyl;
R⁵ is C₁₋₄ alkyl or phenyl, wherein the alkyl is optionally substituted with from 1 to 5 substituents each of which is independently halogen, —O—C₁₋₄ alkyl, or —O-C₁₋₄ haloalkyl; the phenyl is optionally substituted with from 1 to 4 substituents each of which is independently halogen, —C₁₋₄ alkyl, —C₁₋₄ haloalkyl, —O-C₁₋₄ alkyl, or —O—C₁₋₄ haloalkyl; or
alternatively R⁴ and R⁵ together with the —NSO₂— moiety to which they are attached form a sultam group of formula: 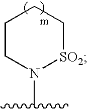
   wherein m is an integer equal to zero, 1, or 2; and the sultam group is optionally substituted with from 1 to 3 substituents each of which is independently halogen, —C₁₋₄ alkyl, —C₁₋₄ haloalkyl, —O—C₁₋₄ alkyl, or —O—C₁₋₄ haloalkyl.

In an aspect of each of the embodiments of the process set forth in the preceding paragraph, R⁴ is —H or —C₁₋₄ alkyl, and R⁵ is C₁₋₄ alkyl; or alternatively R⁴ and R⁵ together with the —NSO₂— moiety to which they are attached form a sultam group of formula: 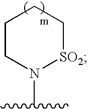
wherein m is an integer equal to zero, 1, or 2.

In a feature of each of the preceding aspects, R⁴ and R⁵ together with the —NSO₂— moiety to which they are attached form a sultam group of formula: 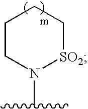
wherein m is an integer equal to zero, 1, or 2.

It is understood that m=zero means that a direct single bond exists between the ring carbon atoms that would otherwise have been indirectly attached to each other via the —(CH₂)ₘ— moiety. For example, when m is zero, the sultam group described in the preceding paragraph is:

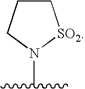

Other embodiments of the process of the invention include the process as originally defined above or as defined in any of the preceding embodiments, wherein R⁶ is —H or —C₁₋₄ alkyl; or R⁶ is —H or —C₁₋₃ alkyl; or R⁶ is —H.

Other embodiments of the process of the invention include the process as originally defined above or as defined in any of the preceding embodiments, wherein R⁷ is —H, —C₁₋₄ alkyl, phenyl, or benzyl; or R⁷ is —H or —C₁₋₄ alkyl; or R⁷ is —C₁₋₄alkyl; or R⁷ is —H, methyl, or ethyl; or R⁷ is methyl or ethyl; or R⁷ is —H; or R⁷ is methyl; or R⁷ is ethyl.

Other embodiments of the process of the invention include the process as originally defined above or as defined in any of the preceding embodiments, wherein each R^{a} is independently —H or —C₁₋₄ alkyl; or is —H or —C₁₋₃ alkyl; or is —H, methyl, or ethyl; or is —H; or is methyl; or is ethyl.

Other embodiments of the process of the invention include the process as originally defined above or as defined in any of the preceding embodiments, wherein each R^{b} is independently —C₁₋₄ alkyl; or is —C₁₋₃ alkyl; or is methyl or ethyl; or is methyl; or is ethyl.

Other embodiments of the process of the invention include the process as originally defined above or as defined in any of the preceding embodiments, wherein R^{c} and R^{d} are each independently —H or —C₁₋₄ alkyl which is optionally substituted with from 1 to 5 substituents each of which is independently halogen, —O—C₁₋₄ alkyl, or —O—C₁₋₄ haloalkyl; or R^{c} and R^{d} are each independently methyl, ethyl, trifluoromethyl; or R^{c} and R^{d} are both methyl.

Other embodiments of the process of the invention include the process as originally defined above or as defined in any of the preceding embodiments, wherein R^{e} is —C₁₋₄ alkyl, —O-C₁₋₄ alkyl, phenyl, or —O-phenyl; wherein the alkyl is optionally substituted with from 1 to 5 substituents each of which is independently halogen, —O—C₁₋₄ alkyl, or —O—C₁₋₄ haloalkyl; and the phenyl is optionally substituted with from 1 to 5 substituents each of which is independently halogen, —C₁₋₄ alkyl, -C₁₋₄ haloalkyl, —O-C₁₋₄ alkyl, —O—C₁₋₄ haloalkyl, —CN, —C(═O)-C₁₋₄ alkyl, —CO₂—C₁₋₄ alkyl, —S—C₁₋₄ alkyl, —N(—C₁₋₄ alkyl)₂, or —C(═O)N(—C₁₋₄ alkyl)₂.

The solvent employed in the condensation reaction of Step C can be any organic compound which under the reaction conditions employed is in the liquid phase, is chemically inert, and will dissolve, suspend, and/or disperse the reactants so as to bring the reactants into contact and permit the reaction to proceed. The solvent is suitably a polar aprotic solvent. Suitable solvents includes nitrites, tertiary amides, ureas, ethers, N-alkylpyrrolidones, pyridines, halohydrocarbons, and esters. Exemplary solvents include acetonitrile, propionitrile, DNF, N,N-dimethylacetamide, DMPU, DMEU, THF, MGBE, ethyl ether, dioxane, 1,2-dimethoxyethane, N-methylpyrrolidone, N-ethylpyrrolidone, pyridine, 2- or 3- or 4-picoline, 2,4,6-collidine, carbon tetrachloride, chloroform, methylene chloride, 1,2-dichloroethane, 1,1,2-trichloroethane, 1,1,2,2-tetrachloroethane, methyl acetate, ethyl acetate, and isopropyl acetate.

In one embodiment, the solvent is selected from the group consisting of nitrites, tertiary amides (e.g., N,N-dialkylamides), ethers, N-alkylpyrrolidones, and pyridines. In another embodiment, the solvent is selected from the group consisting of acetonitrile, propionitrile, DMF, N,N-dimethylacetamide, pyridine, 2-picoline, 3-picoline, 4-picoline, and 2,4,6-collidine.

Step C is suitably conducted at a temperature in the range of from about 20 to about 300° C., and is typically conducted at a temperature in the range of from about 70 to about 150° C. (e.g., from about 90 to about 150° C.). In one embodiment, the temperature is in the range of from about 85 to about 130° C. (e.g., from about 90 to about 125° C.).

The copper promoter in Step C is suitably copper metal, a copper oxide, or a copper salt selected from the group consisting of copper sulfides, halides, sulfonates, alkoxides, carbonates, carboxylates, sulfates, sulfites, thiocyanates, and nitrates. Exemplary copper promoters include CuO, Cu₂O, CuS, Cu₂S, CuCl, CuBr, CuI, CuCl₂, CuBr₂, CuI₂, CuCO₃, CuSO₄, Cu₂SO₄, CuSO₃, Cu(acetate)₂, CuOTf and Cu(SCN)₂. In one embodiment, the copper promoter is copper metal, cuprous oxide (Cu₂O), cuprous chloride (CuCl), cuprous bromide (CuBr), cuprous iodide (CuI), or copper(I) trifluoromethanesulfonate (CuOTf).

The copper chelating agent can be any organic compound that binds to copper by multiple coordinate bonding between two or more electron-pair-donor groups of the chelating agent and the copper as an electron pair acceptor. The chelating agent is suitably a polyamine, a polyaminocarboxylic acid, or a fused or singly bonded bipyridyl compound. Suitable polyamines include the ethylene polyamines (e.g., ethylenediamine, diethylenetriamine, and triethylenetetramine) and 1,2-diaminocycloalkanes (e.g., 1,2-diaminocyclohexane). Suitable polyaminocarboxylic acids include the carboxylic acid derivatives of the ethylene polyamines such as EDTA and carboxylic acid derivatives of 1,2-diaminocycloalkanes such as 1,2-diaminocyclohexanetetracetic acid. Suitable bipyridyl compounds include 2,2′-bipyridyl and 1,10-phenanthroline. In one embodiment of the present invention, the copper chelating agent is 2,2′-bipyridyl, ethylenediamine, 1,2-aminocyclohexane, or 1,10-phenanthroline.

Sulfonamide IV can be employed in Step C in any proportion with respect to Compound IIIa or IIIb which will result in the formation of at least some of Compound Va or Vb. Typically, however, the reactants are employed in proportions which can optimize conversion of at least one of the reactants. In one embodiment, sulfonamide IV is employed in Step C in an amount of from about 0.5 to about 5 equivalents per equivalent of Compound IIIa or IIIb. In another embodiment, the sulfonamide IV is employed in an amount of from about 0.8 to about 3 equivalents per equivalent of Compound IIIa or IIIb. In still another embodiment, the amount of sulfonamide IV employed in Step C is from about 0.9 to about 2 equivalents (e.g., from about 1.0 to about 1.2 equivalents) per equivalent of Compound IIIa or IIIb.

The copper promoter is suitably employed in Step C in an amount in the range of from about 0.1 to about 10 equivalents per equivalent of Compound IIIa or IIIb. In one embodiment, the amount of copper promoter is in the range of from about 0.5 to about 5 equivalents per equivalent of Compound IIIa or IIIb. In another embodiment, the amount of copper promoter is in the range of from about 0.9 to about 3 equivalents (e.g., from about 1 to about 1.2 equivalents) per equivalent of IIIa or IIIb.

The copper chelating agent is suitably present in Step C in a ratio of equivalents of copper chelating agent to copper promoter in the range of from about 1:2 to about 2:1. The ratio of equivalents of copper chelating agent to copper promoter in Step C is typically in the range of from about 1:1.2 to about 1.2:1 (e.g., about 1:1).

The Step C reaction can be conducted by charging the solvent, sulfonamide IV, Compound IIIa or IIIb, copper promoter and copper chelating agent to a suitable reaction vessel, bringing the resulting mixture to reaction temperature, and maintaining the mixture at reaction temperature until the reaction is complete or the desired degree of conversion of the reactants is achieved. The order of addition of the reactants and reagents to the reaction vessel is not critical; i.e., they can be charged concurrently or sequentially in any order. The reaction is generally conducted under an inert atmosphere (e.g., nitrogen or argon gas). The reaction time can vary widely depending upon, inter alia, the reaction temperature and the choice and relative amounts of reactants and promoter, but the reaction time is typically in the range of from about 0.5 to about 24 hours. The product Va or Vb can be subsequently isolated (alternatively referred to as recovered) by, for example, diluting the product mixture with an organic solvent (e.g., chloroform), washing the diluted mixture with an aqueous salt solution, separating the organic and aqueous phases, and recovering Compound Va or Vb from the organic phase. In one embodiment, when a Cu(I) promoter is employed in the Step C reaction, the copper can be removed post-reaction by diluting the product mixture with organic solvent (e.g., CHCl₃), adding an aqueous solution of disodium EDTA, and then oxidizing Cu(I) to Cu(II) with air or H₂O₂ to form a water-soluble blue Cu(II) EDTA complex.

In Step D1, Compound Va is treated with a phenol deprotecting agent to remove the phenol protective group G and thereby obtain Compound VI. Similarly, in Step E, Compound Vb is treated with a phenol deprotecting agent to obtain Compound VIII. As noted above, the —OG group on Compound Va or Vb can be an ether, a silyl ether, a carboxylic ester, a carbonate, a phosphinate or a sulfonate. These —OG groups can be formed by treating the hydroxynaphthyridine precursors of the compounds with phenol protecting agents. Suitable protecting agents and treatment methods are described below in the discussion of Step B. Ether and silyl ether protective groups can subsequently be removed by treatment with acidic reagents including mineral, halogen, and Lewis acids. Suitable acids include HCl, HBr, HF, sulfuric acid, nitric acid, triflic acid (TfOH), trifluoroacetic acid (TFA), acetic acid, BF₃, and BCl₃. Cleavage conditions (e.g., temperature, choice and concentration of acid) can vary from mild to harsh depending upon the lability of the ether protective group. The acyl, sulfonyl, carbonate, and phosphinyl ester protective groups can often be removed via acid or base hydrolysis (e.g., cleavage by treatment with an aqueous alkali metal hydroxide such as NaOH or an aqueous alkoxide such as NaOMe or NaOEt), although occasionally other means (e.g., hydrogenolysis) may need to be employed. Further description of phenol deprotecting agents and deprotection treatments suitable for use in either Step D1 or Step E can be found in Protective Groups in Organic Chemistry, edited by J. F. W. McOmie, Plenum Press, New York, 1973, pp. 145-182; and in T. W. Greene and P. G. M. Wuts, Protective Groups in Organic Synthesis, 2^{nd} edition, John Wiley, New York, 1991, pp. 143-174.

Step D2 concerns the coupling of Compound VI with amine VII to obtain, Compound VIII. The coupling reaction is suitably conducted in solvent at a temperature in the range of from about 40 to about 200° C., and is typically conducted at a temperature in the range of from about 50 to about 160° C. Suitable solvents include aliphatic hydrocarbons, alicyclic hydrocarbons, aromatic hydrocarbons, halogenated aliphatic hydrocarbons, alcohols, esters, ethers, and nitrites. Exemplary solvents include pentane, hexane, carbon tetrachloride, chloroform, methylene chloride, 1,2-dichloroethane, 1,1,2-trichloroethane, 1,1,2,2-tetrachloroethane, cyclohexane, toluene, o- and m- and p-xylene, ethylbenzene, methanol, ethanol, isopropanol, n-butanol, t-butyl alcohol, ethyl ether, ABE, THF, dioxane, 1,2-dimethoxyethane, anisole, phenetole, methyl acetate, ethyl acetate, isopropyl acetate, acetonitrile, and propionitrile. In one embodiment, the solvent is selected from the group consisting of C₃-C₁₀ linear and branched alkanes, C₁-C₁₀ linear and branched halogenated alkanes, C₅-C₁₀ cycloalkanes, C₆-C₁₄ aromatic hydrocarbons, dialkyl ethers wherein each alkyl is independently a C₁-C₆ alkyl, C₁-C₆ linear and branched alkanes substituted with two —O—C₁-C₆ alkyl groups (which are the same or different), C₄-C₈ cyclic ethers and diethers, C₆-C₈ aromatic ethers, C₂-C₆ aliphatic nitrites, and C₁-C₆ alkyl esters of C₁-C₆ alkylcarboxylic acids. In another embodiment, the solvent is selected from alcohols, esters and ethers. In an aspect of the preceding embodiment, the solvent is selected from the group consisting of C₁-C₆ alkyl alcohols, dialkyl ethers wherein each alkyl is independently a C₁-C₄ alkyl, C₄-C₅ cyclic ethers, and C₁-C₄ alkyl esters of C₁-C₄ alkylcarboxylic acids. In another aspect of the preceding embodiment, the solvent is methanol, ethanol, n-propanol, isopropanol, t-butyl alcohol, diethylether, 1,2-dimethoxyethane, THF, methyl acetate, ethyl acetate, or isopropyl acetate.

Amine VII can be employed in Step D2 in any proportion which will result in the formation of at least some of Compound VIII. Typically, however, the reactants are employed in proportions which can optimize conversion of at least one of the reactants, and usually amine VII is employed in an amount that can optimize the conversion of Compound VI. In one embodiment, amine VII is employed in an amount of from about 0.5 to about 10 equivalents (e.g., from about 1 to about 10 equivalents) per equivalent of Compound VI. It is preferred to use an excess of amine in order to increase the degree of conversion and/or shorten the reaction time. Accordingly, in other embodiments, amine VII is employed in an amount of from about 1.1 to about 10 equivalents, or an amount of from about 2 to about 10 equivalents, or an amount of from about 2 to about 5 equivalents, or from about 2.5 to about 3.5 equivalents (e.g., about 3 equivalents), per equivalent of Compound VI.

The reaction of Step D2 is suitably conducted by adding amine VII to a solution or suspension of Compound VI in the selected solvent and then heating the mixture to reaction temperature and maintaining at reaction temperature until the reaction is complete or the desired degree of conversion of the reactants is achieved. When Compound VII is a carboxylic acid (i.e., when R⁷=H), the coupling reaction is typically conducted in the presence of a dehydrating/condensing agent such as dicyclohexylcarbodiimide, EDC, or 2-chloropyridinium iodide, in order to promote conversion to the amide and minimize back reaction to the acid. Alternatively, the acid-amine coupling reaction can be conducted with concurrent removal (e.g., by distillation into a trap) of the water by-product. Recovery and isolation of the amide product can be accomplished using conventional procedures.

Amines of Formula (VII) can be prepared using the methods described in Richard Larock, Comprehensive Organic Transformations, VCH Publishers Inc, 1989, pp 385-438, or as described in Morrison and Boyd, Organic Chemistry, 4^{th} edition, Allyn and Bacon, 1983, pp. 893-897, or routine variations thereof.

The present invention includes a process for preparing a compound of Formula (VIII) which comprises Steps C, D and E as described above and which further comprises:
(B) treating a compound of Formula (IIa) or (IIb): 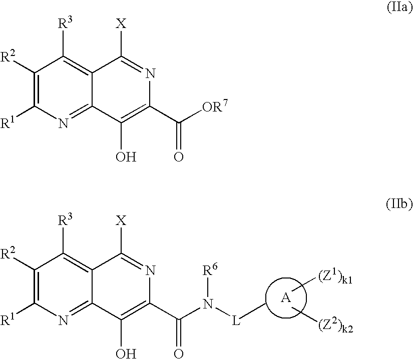
   with a phenol protecting agent to obtain Compound IIIa or IIIb. It is understood that embodiments of this process include the Steps B. C, D and, E as originally described above, incorporating one or more embodiments, aspects, or features of one or more of Steps C, D and E as set forth above and/or incorporating one or more embodiments, aspects or features of Step B as set forth below.

Suitable protecting agents include compounds selected from the group consisting of:
(i) a compound of formula G^{a}-Q, wherein Q is halide and G^{a} is:
   (1)—C₁₋₆ alkyl,
   (2) —C₁₋₆ alkyl-aryl,
   (3) —C₁₋₆ alkyl-O-C₁₋₆ alkyl,
   (4)-C₁₋₆ alkyl-O-C₁₋₆ alkyl-aryl,
   (5) —C₃₋₈ cycloalkyl,
   (6) allyl,
   (7)-Si(C₁₋₆ alkyl)ₙ(aryl)₃₋ₙ, wherein n is an integer equal to zero, 1, 2, or 3;
   (8) —C(═O)—C₁₋₆ alkyl,
   (9) —C(═O)-aryl,
   (10) —C(═O)-C₁₋₆ alkyl-aryl,
   (11) —C(═O)—O—C₁₋₆ alkyl,
   (12) —C(═O)—O-aryl,
   (13) —C(═O)—O—C₁₋₆ alkyl-aryl,
   (14) —SO₂—C₁₋₆ alkyl,
   (15) —SO₂—C₁₋₆ haloalkyl, or
   (16) —SO₂-aryl,
(ii) a sulfate compound of formula (G^{b})₂SO₄, wherein G^{b} is —C₁₋₆ alkyl or —C₁₋₆ alkyl-aryl; and
(iii) an anhydride of formula (G^{c})₂O, wherein G^{c} is —C(═O)—C₁₋₆ alkyl, —C(═O)-aryl, or —C(═O)—C₁₋₆ alkyl-aryl;
(iv) a diazo compound of formula G^{d}-N₂, wherein G^{d} is —C₁₋₆ alkylidenyl, —C₁₋₆ alkylidenyl-aryl or —C₃₋₈ cycloalkylidenyl;
wherein each aryl in (i), (iii), or (iv) is optionally substituted with from 1 to 5 substituents each of which is independently halogen, —C₁₋₄ alkyl, —O—C₁₋₄ alkyl, or nitro;
and wherein treatment with G^{a}-Q, (G^{b})₂SO₄, or (G^{c})₂O results in the attachment of G^{a}, G^{b}, or G^{c} as the phenol protective group G in Compound IIIa or IIIb, and treatment with G^{d}-N₂ results in the attachment of G^{d}H as the phenol protective group G in Compound IIIa or IIb.

Treating either Compound IIa or IIb with a compound of formula G^{a}-Q, typically in the presence of base, will result in the alkylation, acylation or sulfonylation of the hydroxy group on the naphthyridine ring (also referred to herein as the “phenolic group”) to give the corresponding ether or ester group —OG^{a} (i.e., protective group G in Compound IIIa or IIIb =G^{a}). Reaction of the phenolic group with the alkyl or aralkyl sulfate (G^{b})₂SO₄, typically in the presence of a base, will form the phenolic ether group —OG^{b} (i.e., protective group G in Compound IIIa or IIIb=G^{b}). Treatment of the phenolic group with the anhydride (G^{c})₂O results in the acylation of the phenolic group to form the phenolic ester group —OG^{c} (i.e., protective group G in Compound IIIa or IIIb =G^{c}). Reaction of the phenolic group with azo compound G^{d}-N2 will result in the formation of the phenolic ether group —OG^{d}H (i.e., the alkylidenyl, aryalkylidenyl, and cycloalkylidenyl in the azo compound respectively result in the formation of an alkyl, arylalkyl, and cycloalkyl phenolic ether, such that the protective group G in Compound IIIa or IIIb =G^{d}H). Further description of these and other phenol protecting agents suitable for use in Step B can be found in Protective Groups in Organic Chemistry, edited by J. F. W. McOmie, Plenum Press, New York, 1973, pp. 145-182; and in T. W. Greene and P. G. M. Wuts, Protective Groups in Organic Synthesis, 2^{nd} edition, John Wiley, New York, 1991, pp. 143-174; the disclosures of which are hereby incorporated by reference in their entireties.

The phenolic group in Compound IIa can also be protected by the formation of an alkylenedioxy group; i.e., Compound IIa can be treated with a carbonyl-containing compound of Formula (IX): 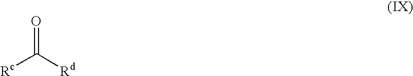
in the presence of an acid to form a compound of Formula (X): 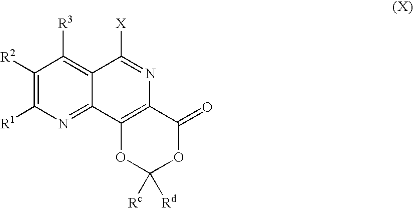
wherein R^{c} and R^{d} are as heretofore defined. In one embodiment, Compound IX is acetone (or, alternatively, an “acetone equivalent” such as 2,2-dimethoxypropane or 2-methoxypropene) and the protective group in Compound X is an acetonide. Acids which can be employed in the treatment of Compound IIa with IX include HCl, HBr, sulfuric acid, methanesulfonic acid, acetic acid, and TFA. The acid is suitably employed in a catalytic amount, a stoichiometric amount or an excess amount with respect to the amount of Compound IX. The alkylene dioxy protective group can subsequently be removed (e.g., from Compound Va or Vb) by acid or base hydrolysis.

The phenolic group in Compound IIa can also be protected by the formation of a borate ester group; i.e., Compound IIa can be treated with a boronic acid alone or in the presence of an acid or base. In one embodiment, a compound of Formula (XIa) or (XIb): 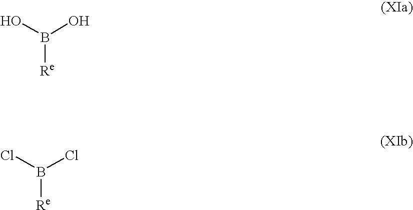
can be employed to treat Compound IIa in the presence of an acid or base to form a compound of Formula (XII): 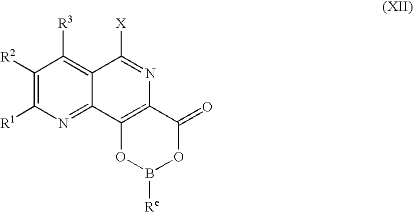
wherein R^{e} is as heretofore defined. The protective group can subsequently be removed by acid or base hydrolysis.
The present invention includes a process for preparing a compound of Formula (VIII) which comprises Steps B, C, D and E as described above and which further comprises:
(A) contacting a compound of Formula (Ia) or (Ib): 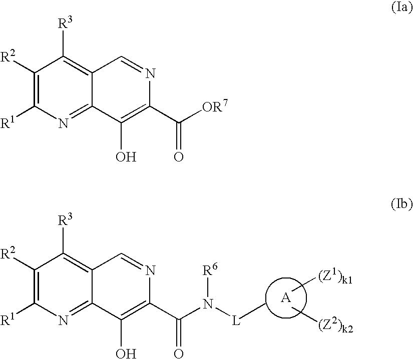
   with a halogenating agent to obtain Compound (IIa) or (IIb). It is understood that embodiments of this process include the Steps A, B, C, D and E as originally described above, incorporating one or more embodiments, aspects, or features of one or more of Steps B, C, D and E as set forth above and/or incorporating one or more embodiments, aspects or features of Step A as set forth below.

Halogenating agents suitable for use in Step A include halogens, interhalogen compounds, hypohalite salts or esters, phosphoryl halides, oxalyl halides, haloamides, haloureas, halocarbamates, halosulfonamides, halosuccinimides, and halohydantoins. Exemplary halogenating agents include Cl₂, Br₂, ICl, IBr, POCl₃, POBr₃, NaOCl, NaOBr, oxalyl chloride, sulfonyl chloride, N-bromosuccinimide, N-chlorosuccinimide, N-iodosuccinimide, 1,3-dichloro-5,5-dimethylhydantoin, and 1,3-dibromo-5,5-dimethylhydantoin. In one embodiment of Step A, the halogenating agent is selected from the group consisting of N-halosuccinimides and halohydantoins. In an aspect of this embodiment, the halogenating agent is N-bromosuccinimide and 1,3-dibromo-5,5-dimethylhydantoin.

The halogenation in Step A is typically conducted in a solvent, which may be any organic compound which under the halogenation conditions employed is in the liquid phase, is chemically inert, and will dissolve, suspend, and/or disperse the reactants so as to permit the halogenation to proceed. Suitable solvents include ethers, esters, halogenated aliphatic hydrocarbons, halogenated aromatic hydrocarbons, alcohols, nitriles, and tertiary amides. In one embodiment the solvent is selected from the group consisting of chlorinated aliphatic hydrocarbons, ethers and esters. In another embodiment, the solvent is selected from the group consisting of C₁-C₁₀ linear and branched halogenated alkanes, halogenated C₆-C₁₄ aromatic hydrocarbons, dialkyl ethers wherein each alkyl is independently a C₁-C₆ alkyl, C₁-C₆ linear and branched alkanes substituted with two —O—C₁-C₆ alkyl groups (which are the same or different), C₄-C₈ cyclic ethers and diethers, C₆-C₈ aromatic ethers, C₁-C₆ alkyl esters of C₁-C₆ alkylcarboxylic acids, C₁-C₁₀ alkyl alcohols, C₂-C₆ aliphatic nitriles, C₇-C₁₀ aromatic nitriles, and N,N-di-(C₁-C₆ alkyl) C₁-C₆ alkylcarboxylic acid amides. Exemplary solvents include carbon tetrachloride, chloroform, methylene chloride, 1,2-dichloroethane (DCE), 1,1,2-trichloroethane (TCE), 1,1,2,2-tetrachloroethane, mono- and di- and tri-chlorobenzenes, ethyl ether, MTME, TB, dioxane, 1,2-dimethoxyethane (DME), anisole, phenetole, methyl acetate, ethyl acetate, isopropyl acetate, ethanol, n- and iso-propanol, tert-butyl alcohol, tert-amyl alcohol, acetonitrile, propionitrile, benzonitrile, p-tolunitrile, DMF, and N,N-dimethylacetamide.

The halogenation in Step A is suitably conducted at a temperature in the range of from about −80 to about 150° C., and is typically conducted at a temperature in the range of from about 10 to about 60° C.

The halogenation in Step A can be conducted by adding the halogenation agent (e.g., a halosuccinimide) to Compound Ia or Ib dissolved, dispersed or suspended in solvent, and then bringing the mixture to reaction temperature and maintaining at reaction temperature until the halogenation is complete or the desired degree of conversion of the reactants is achieved.

In a further embodiment, the compound employed in Step A is Compound Ib, and the process further comprises coupling Compound Ia with an amine of Formula (VII), as heretofore defined. The procedure and conditions for coupling amines of Formula (VII) with Compound VI set forth above in the description of Step D2 (including the choice of solvent, reaction temperature, and relative amounts of reactants) can be employed in an analogous manner to the coupling of amine VII with Compound Ia.

Compounds of Formula (Ia) can be prepared as follows:

In Scheme B, following the procedure set forth in Ornstein et al., J. Med. Chem. 1989, 32: 827-833, a cyclic anhydride such as quinolinic anhydride (i.e., R¹=R²=R³=H in B-1) can be opened with isopropanol to provide mono acid B-2, which can be converted to the corresponding acyl chloride B-3 (e.g., by refluxing thionyl chloride). Acyl chloride B-3 can then be reduced to the corresponding alcohol B-4, which can be converted to the corresponding bromide through the action of bromine in the presence of triphenylphosphine. Alkylation of the bromide with the sodium anion of phenylsulfonamide B-5 in a polar aprotic solvent like DMF can provide sulfonamide B-6, which can be treated with a base (e.g., alkali metal alkoxide such as sodium methoxide) to provide the bicyclic ester B-7 via a Dieckmann cyclization. Saponification of the ester (e.g., with aqueous NaOH at reflux) will afford the corresponding acid. Transesterification of B-7 or esterification of B-8 can afford other esters embraced by Formula (Ia).

The starting anhydrides of formula B-1 can be prepared via methods described in Philips et al., Justus Liebigs Ann. Chem. 1895, 288: 2535; Bernthsen et al., Chem. Ber. 1887; 20: 1209; Bly et al., J. Org. Chem. 1964, 29: 2128-2135; and Krapcho et al., J. Heterocycl. Chem. 1993, 30: 1597-1606; or routine variations thereof.

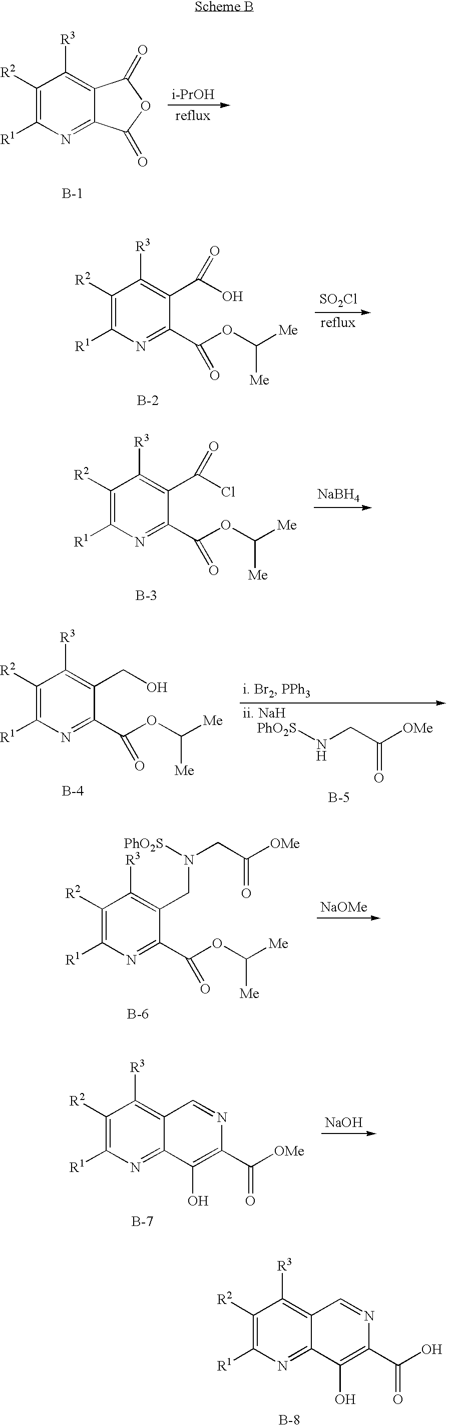

Scheme C depicts an alternative synthesis in which alcohol B-4 can undergo the Mitsunobu reaction with the phenylsulfonamide of glycine methyl ester to provide B-6. The sulfonamide B-6 can again be elaborated to provide the acid B-8, or esters thereof.

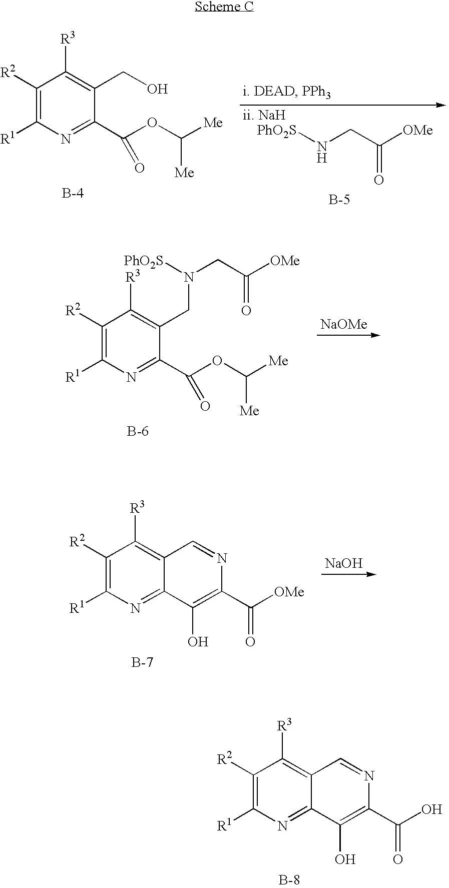

The present invention also includes a process for preparing a compound of Formula (VIII′): 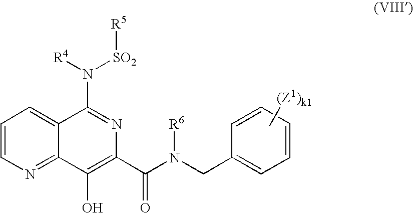
which comprises:
(C) reacting a compound of Formula (IIIa′) or (IIIb′): 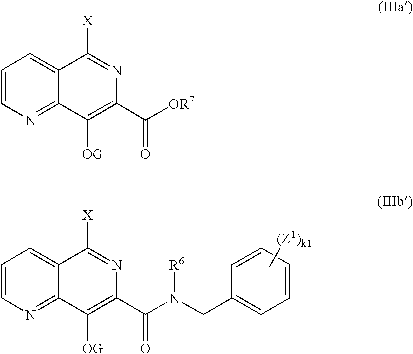
   with a sulfonamide of Formula (IV):
   R⁵SO₂—NHR⁴ (IV)
   in solvent and in the presence of a copper promoter and a copper chelating agent to obtain a compound of Formula (Va′) or (Vb′): 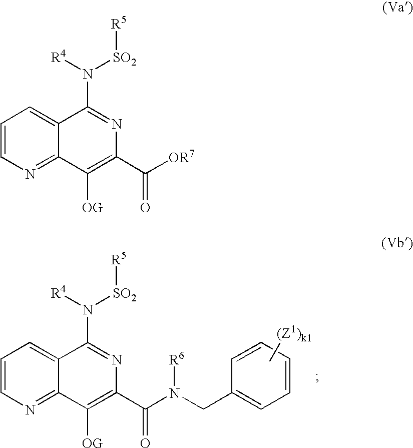
(D) when the compound resulting from Step C is Compound Va′,
   (D1) treating Compound Va′ with a phenol deprotecting agent to obtain a compound of Formula (VI′): 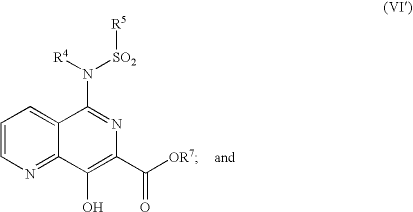
   (D2) coupling Compound VI′ with an amine of Formula (VII′): 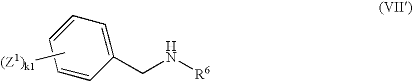
to obtain Compound VIII′; and
(E) when the compound resulting from Step C is Compound Vb′, reacting Compound Vb′ with a phenol deprotecting agent to obtain Compound VIII′;
   wherein G, X, Z¹, k1, R⁴, R⁵, R⁶, and R⁷ are each as originally defined above in the

### SUMMARY OF THE INVENTION

An embodiment of the process for preparing Compound VIII′ is a process which comprises
(C) reacting a compound of Formula (IIIa′): 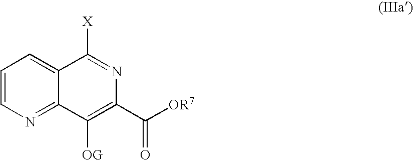
   with a sulfonamide of Formula (IV):
   R⁵SO₂—NHR⁴ (IV)
   in solvent and in the presence of a copper promoter and a copper chelating agent to obtain a compound of Formula (Va′): 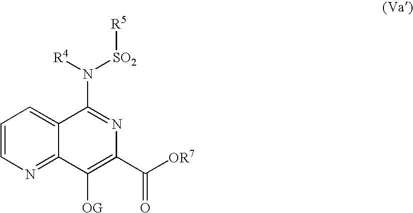
   (D1) treating Compound Va′ with a phenol deprotecting agent to obtain a compound of Formula (VI′):

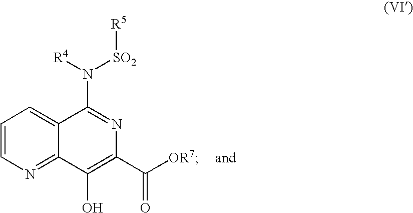

(D2) coupling Compound VI′ with an amine of Formula (VII′): 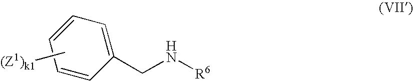
to obtain Compound VIII′.

Another embodiment of the process for preparing Compound VIII′ is a process which comprises:

(C) reacting a compound of Formula (IIIb′): 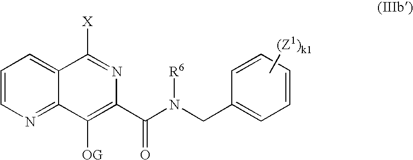
with a sulfonamide of Formula (IV):
R⁵SO₂—NHR⁴ (IV)
in solvent and in the presence of a copper promoter and a copper chelating agent to obtain a compound of Formula (Vb′): 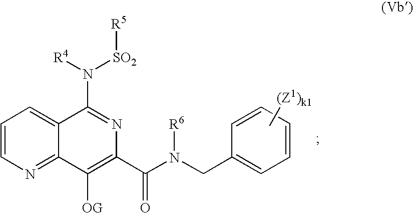
(E) reacting Compound Vb′ with a phenol deprotecting agent to obtain Compound VIII′.

An aspect of the process for preparing Compound VIII′ is the process as defined above or as set forth in either of the two preceding embodiments, wherein the solvent in Step C is a polar aprotic solvent selected from the group consisting of nitrites, tertiary amides, ureas, ethers, N-alkylpyrrolidones, pyridines, halohydrocarbons, and esters;
the sulfonamide IV is employed in Step C in the amount of from about 0.8 to about 3 equivalents per equivalent of Compound IIIa′ or IIIb′; and

Step C is conducted at a temperature in the range of from about 70 to about 150° C.

A further aspect of the process for preparing Compound VIII′ is the process as defined above or as set forth in either of the two preceding embodiments, and optionally also incorporating the preceding aspect, wherein
the copper promoter in Step C is copper metal, a copper oxide, or a copper salt selected from the group consisting of copper sulfides, halides, sulfonates, alkoxides, carbonates, carboxylates, sulfates, sulfites, thiocyanates, and nitrates;
the copper chelating agent in Step C is a polyamine, a polyaminocarboxylic acid, or a fused or singly bonded bipyridyl compound;
the copper promoter is employed in Step C in an amount in the range of from about 0.9 to about 3 equivalents per equivalent of Compound IIIa′ or IIIb′; and
the ratio of equivalents of copper chelating agent to copper promotor in Step C is in the range of from about 1:1.2 to about 1.2:1.

Additional embodiments of the process for preparing Compound VII′ include the process as defined above or as defined in either of the two preceding embodiments thereof and incorporating any one or more of the embodiments set forth earlier in the process for preparing Compound VIII, such as the embodiments restricting the definitions of one or more of G, T, T′, X, Y, Z¹, k1, R⁴, R⁵, R⁶, R⁷, R^{a}, R^{b}, R^{c}, and R^{d}; and/or describing one or more reaction conditions, and/or describing one or more reagents (e.g., the copper promoter, chelating agent, solvent, or the like).

The process for preparing Compound VIII′ can be conducted in a manner analogous to that described earlier for the process for preparing Compound VIII.

The present invention also includes a process for preparing a compound of Formula (VIII″): 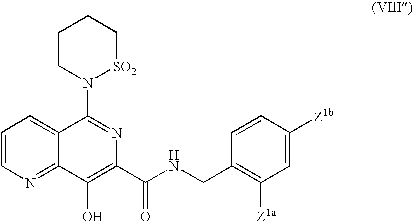
which comprises:
(C) reacting a compound of Formula (IIIa′): 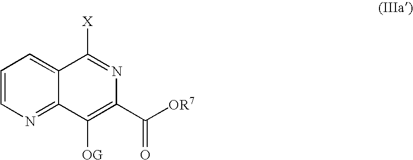
   with sultam 4: 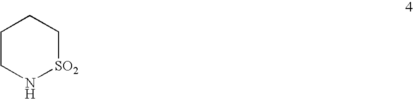
   in solvent and in the presence of a copper promoter and a copper chelating agent to obtain a compound of Formula (Va″): 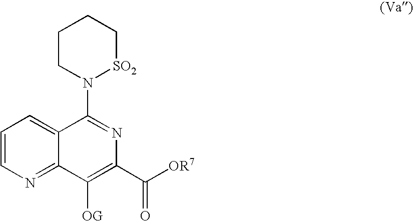
(D1) treating Compound Va″ with a phenol deprotecting agent to obtain a compound of Formula (VI″): 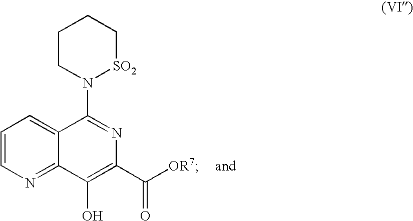
   (D2) coupling Compound VI″ with an amine of Formula (VII″) 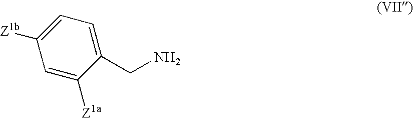
      to obtain Compound VII″; wherein:
G is a phenol protective group;
X is halo; Z₁ₐ and Z_{1b} are each independently —H or halo; and
R⁷ is —C₁₋₆ alkyl, phenyl, or benzyl.

Embodiments of the process for preparing Compound VIII″, include the process as just described incorporating any one or more of the following features:
the —OG group on Compound III′ and Va″ is an ether, a silyl ether, a carboxylic ester, or a sulfonate;
G is —SO₂—C₁₋₆ alkyl, —SO₂—C₁₋₆ haloalkyl, or —SO₂-aryl, wherein the aryl is optionally substituted with from 1 to 5 substituents each of which is independently halogen, —C₁₋₄ alkyl, —O—C₁₋₄ alkyl, or nitro;
G is CH₃SO₂—, CF₃SO₂—, or p-toluenesulfonyl;
G is p-toluenesulfonyl;
X is Cl or Br;
R⁷ is —C₁₋₄ alkyl;
one of Z^{1a} and Z^{1b} is fluoro or chloro, and the other of Z^{1a} and Z^{1b} is H, fluoro, or chloro;
amine VII″ in Step D2 is Compound 10: 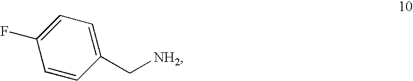
   so that
   Compound VIII″ is Compound 15;
the solvent in Step C is a polar aprotic solvent selected from the group consisting of nitrites, tertiary amides, ethers, N-alkylpyrrolidones, and pyridines;
Step C is conducted at a temperature in the range of from about 70 to about 150° C.;
the copper promoter in Step C is copper metal, a copper oxide, or a copper salt selected from the group consisting of copper sulfides, halides, sulfonates, alkoxides, carboxylates, sulfates, thiocyanates, and nitrates;
the copper chelating agent in Step C is a polyamine, a polyaminocarboxylic acid, or a fused or singly bonded bipyridyl compound;
the copper promoter is employed in Step C in an amount in the range of from about 0.9 to about 3 equivalents per equivalent of Compound IIIa′;
the ratio of equivalents of copper chelating agent to copper promotor in Step C is in the range of from about 1:1.2 to about 1.2:1; and
the sultam 4 is employed in Step C in the amount of from about 0.8 to about 3 equivalents per equivalent of Compound IIIa′.

The present invention further includes a process for preparing Compound VIII″ which comprises:
(C) reacting a compound of Formula (IIIb″): 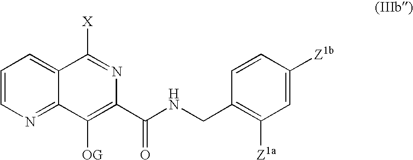
   with sultam 4: 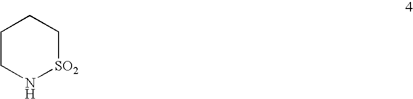
   in solvent and in the presence of a copper promoter and a copper chelating agent to obtain a compound of Formula (Vb″): 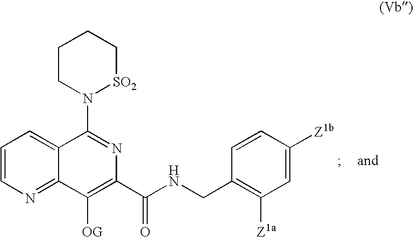
(E) reacting Compound Vb″ with a phenol deprotecting agent to obtain Compound VIII″; wherein:
G is a phenol protective group;
X is halo; and Z^{1a} and Z^{1b} are each independently —H or halo.

Embodiments of the process for preparing Compound VIII″ include the process as just described incorporating any one or more of the following features:
the —OG group on Compound IIIb″ and Vb″ is an ether, a silyl ether, a carboxylic ester, or a sulfonate;
G is —SO₂—C₁₋₆ alkyl, —SO₂—C₁₋₆ haloalkyl, or —SO₂-aryl, wherein the aryl is optionally substituted with from 1 to 5 substituents each of which is independently halogen, —C₁₋₄ alkyl, —O—C₁₋₄ alkyl, or nitro;
G is CH₃SO₂—, CF₃SO₂—, or p-toluenesulfonyl;
G is p-toluenesulfonyl;
X is Cl or Br;
one of Z^{1a} and Z^{1b} is fluoro or chloro, and the other of Z^{1a} and Z^{1b} is H, fluoro, or chloro;
Compound IIIb′ in Step C is: 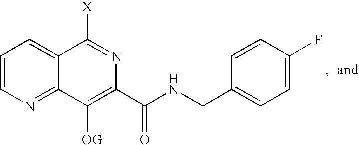
   Compound Vb″ in Step E is 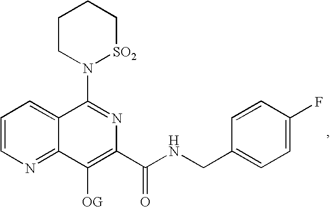
   so that
   Compound VIII″ is Compound 15;
the solvent in Step C is a polar aprotic solvent selected from the group consisting of nitrites, tertiary amides, ethers, N-alkylpyrrolidones, and pyridines;
Step C is conducted at a temperature in the range of from about 70 to about 150° C.;
the copper promoter in Step C is copper metal, a copper oxide, or a copper salt selected from the group consisting of copper sulfides, halides, sulfonates, alkoxides, carboxylates, sulfates, thiocyanates, and nitrates;
the copper chelating agent in Step C is a polyamine, a polyaminocarboxylic acid, or a fused or singly bonded bipyridyl compound;
the copper promoter is employed in Step C in an amount in the range of from about 0.9 to about 3 equivalents per equivalent of Compound IIIb″; and
the ratio of equivalents of copper chelating agent to copper promotor in Step C is in the range of from about 1:1.2 to about 1.2:1; and
the sultam 4 is employed in Step C in the amount of from about 0.8 to about 3 equivalents per equivalent of Compound IIIb″.

The present invention also includes a process for preparing Compound VIII which comprises Step C′ as described below and Steps D and E as originally defined above; i.e., Step C′ as defined below replaces Step C in the process for preparing Compound VII as originally described above in the Summary of the Invention. Step C′ is as follows:
(C′) reacting a compound of Formula (IIIa) or (IIIb): 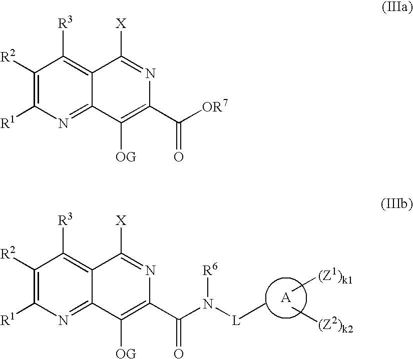
   with a sulfonamide of Formula (IV):
   R⁵SO₂—NHR⁴ (IV)
   in solvent and in the presence of a catalytic amount of a copper (I) compound, a base and optionally a ligand to obtain a compound of Formula (Va) or (Vb): 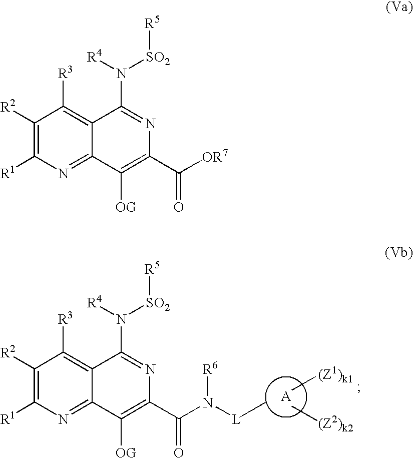
   wherein A, G, R¹, R², R³, R⁴, R⁵, R⁶, R⁷, Z¹, Z², k1 and k2 are all as originally defined above. Embodiments of the process for preparing Compound VIII via Step C′, D and E include all of the earlier-described embodiments or aspects for preparing Compound VIII via Step C in which one or more of the definitions of A, G, R¹, R², R³, R⁴, R⁵, R⁶, R⁷, Z¹, Z², k1 and k2 is restricted. Embodiments of the process for preparing Compound VIII via Step C′ also include embodiments analogous to those involving Step C which further comprise Step B or Step B and Step A as heretofore described. In the same manner, the present invention also includes the processes for preparing Compound VIII′ and for preparing Compound VIII″ in which Step C is replaced with a Step C′ in a manner analogous to that just described with respect to the preparation of Compound VII.

In Step C′, the copper catalyst is an inorganic copper I compound. Suitable catalysts include copper I oxide, sulfide, halides, and thiocyanate. In one aspect, the catalyst is selected from cuprous oxide (Cu₂O) and the cuprous halides (e.g., CuI). The copper I compound is employed in a catalytic amount, and may be employed, for example, in an amount of from about 1 to about 20 mole % (e.g., from about 5 to about 15 mole %, or about 10 mole %), relative to the moles of Compound IIIa or IIIb. The base is typically an inorganic base (e.g., K₃PO₄ and K₂CO₃) and is suitably employed in an amount of from about 0.9 to about 2 equivalents (e.g., from about 1 to about 1.5 equivalents) per equivalent of Compound IIIa or IIIb. Suitable solvents include the solvents described above as suitable for use in Step C.

Suitable ligands for use in Step C′ include bipyridyl, PPh₃, P(o-Tolyl)₃, P(furyl)₃, P(OnBu)₃, P(2,4-di-t-Butylphenoxy)₃, AsPh₃, bis-diphenylphosphinoethane, bis-diphenylphosphinopropane, bis-diphenylphosphinobutane, o-bis-diphenylphosphinobenzene, bis-diphenylphosphinoferrocene, bis-di-o-tolylphosphinoferrocene, bis-diphenylphosphinobinaphthyl, bis-ditolylphosphinobinaphthyl, NH₂CH₂CH₂NH₂, MeNHCH₂CH₂NHMe, Me₂NCH₂CH₂NHMe, MeNHCH₂CH₂CH₂NHMe, 2-pyridyl-CH₂NH₂, 2-pyridyl-CH₂CH₂NH₂, 2-pyridyl-CH₂CH₂NHMe, 2,2′-Binaphthyldiamine, o-phenylene diamine, 1,2-cyclohexanediamine, bis-dimethyloxalamide, 2,2′-bis-4-benzyl-2-oxazoline, 2,2′-bis-4-t-butyl-2-oxazoline, 2,2′-methylenebis-4-phenyl-2-oxazoline, 2,2′-methylenebis-4,5-diphenyl-2-oxazoline, 2,2′-methylenebis-4,5-indano-2-oxazoline, 2,2′-isopropylidenebis4-t-butyl-2-oxazoline, 2,6-bis-(4,5-dihydro-4-isopropyl-2-oxazolyl)pyridine, 2,6-bis-(4,5-dihydro-4-phenyl-2-oxazolyl)pyridine, 2,6-bis-(4,5-dihydro-4-methyl-5-phenyl-2-oxazolyl)pyridine, (DHQ)₂AQN, DHQD) ₂PHAL, (DHQ)₂PHAL, Trost Ligand, Jacobsens Ligand, 1,3-Dimesitylimidasolonium tetrafluoborate, TsNHCH₂CH₂NHTs, TsNHCH₂CH₂CH₂NHTs, TsNHCH₂CH₂CH₂NTsCH₂CH₂CH₂NHTs, TsNHCH₂CH₂NTsCH₂CH₂NHTs, di-p-tosyl-1,2-cyclohexanediamine, 1,2-diphenyletylenediamine, QUINAP, NMe₂CH₂CH₂NMe₂, and 9,10-phenanthroline. These ligands are commercially available (e.g., from Sigma-Aldrich and Strem Chemicals). The ligand is suitably present in Step C′ in an amount of from about 5 to about 50 mole % (e.g., from about 10 to about 25 mole %), relative to the moles of Compound IIIa or IIIb.

Sulfonamide IV is employed in Step C′ in the same proportions with respect to Compound IIIa or IIIb as set forth above for Step C.

Step C′ is suitably conducted at a temperature in the range of from about 20 to about 300° C., and is typically conducted at a temperature in the range of from about 70 to about 150° C. (e.g., from about 90 to about 150° C.). In one embodiment, the temperature is in the range of from about 85 to about 130° C. (e.g., from about 90 to about 125° C.).

The Step C′ reaction is conducted in substantially the same manner as described above for Step C; e.g., the Step C′ reaction can be conducted by charging the solvent, sulfonamide IV, Compound IIIa or IIIb, copper catalyst, base, and optional ligand to a suitable reaction vessel, bringing the resulting mixture to reaction temperature, and maintaining the mixture at reaction temperature until the reaction is complete or the desired degree of conversion of the reactants is achieved. The order of addition of the reactants and reagents to the reaction vessel is not critical; i.e., they can be charged concurrently or sequentially in any order.

The Step C′ reaction has been conducted with the ligands listed above, wherein 0.20 mmol tosyl bromonaphthyridine 7 (see Step 3 of Example 2 below), 0.22 mmol sultam 4, 0.22 mmol K₃PO₄, 0.020 mmol Cu₂O, 0.50 mL DMF and 0.050 mmol of the ligand were heated to 100° C. for 18 hours, and each run was assayed by HPLC. Yields of product 8 were in the range of 50-80%.

An embodiment of particular interest with respect to Step C′ is as follows:

A process for preparing a compound of Formula (VIII′): 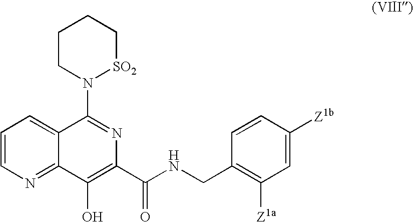
which comprises:
(C) reacting a compound of Formula (IIIa′): 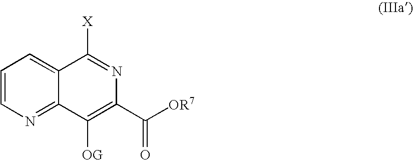
   with sultam 4: 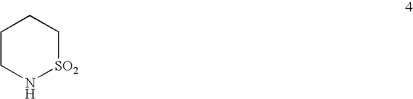
   in solvent and in the presence of a catalytic amount of a copper (I) compound, a base and optionally a ligand to obtain a compound of Formula (Va″): 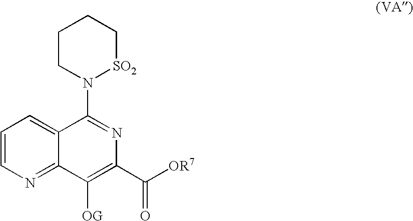
(D1) treating Compound Va″ with a phenol deprotecting agent to obtain a compound of Formula (VI″): 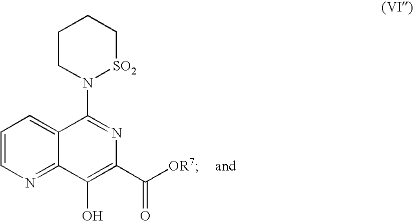
(D2) coupling Compound VI″ with an amine of Formula (VII″): 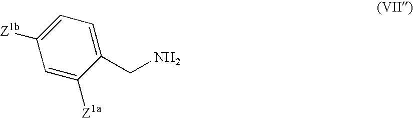
   to obtain Compound VIII″; wherein G is a phenol protective group; X is halo; Z^{1a} and Z^{1b} are each independently —H or halo; and R⁷ is —C₁₋₆ alkyl, phenyl, or benzyl.

Embodiments of the process for preparing Compound VIII″ via Step C′ include the process as just described incorporating any one or more of the following features:
the —OG group on Compound IIIa′ and Va″ is an ether, a silyl ether, a carboxylic ester, or a sulfonate;
G is —SO₂—C₁₋₆ alkyl, —SO₂—C₁₋₆ haloalkyl, or —SO₂-aryl, wherein the aryl is optionally substituted with from 1 to 5 substituents each of which is independently halogen, —C₁₋₄ alkyl, —O-C₁₋₄ alkyl, or nitro;
G is CH₃SO₂—, CF₃SO₂—, or p-toluenesulfonyl;
G is p-toluenesulfonyl;
X is Cl or Br;
R⁷ is —C₁₋₄ alkyl;
one of Z^{1a} and Z^{1b} is fluoro or chloro, and the other of Z^{1a} and Z^{1b} is H, fluoro, or chloro;
amine VII″ in Step D2 is Compound 10: 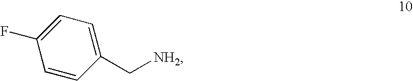
   so that
   Compound VIII″ is Compound 15;
the solvent in Step C is a polar aprotic solvent selected from the group consisting of nitriles, tertiary amides, ethers, N-alkylpyrrolidones, and pyridines;
Step C is conducted at a temperature in the range of from about 70 to about 150° C.;
the base is K₃PO₄; and
the copper catalyst is CuO.

Still other embodiments of the present invention include any of the processes as originally defined and described above and any embodiments or aspects thereof as heretofore defined, further comprising isolating (which may be alternatively referred to as recovering) the compound of interest (e.g., Compound VIII) from the reaction medium.

If desired, the progress of the reaction in any of the above-described chemical reactions can be followed by monitoring the disappearance of a reactant and/or the appearance of the product using LC, HPLC, NMR, or G^{C} .

As used herein, the term “C₁-C₆ alkyl” (which may alternatively be referred to herein “C₁₋₆ alkyl”) as means linear or branched chain alkyl groups having from 1 to 6 carbon atoms and includes all of the hexyl alkyl and pentyl alkyl isomers as well as n-, iso-, sec- and t-butyl, n- and isopropyl, ethyl and methyl. “C₁-C₄ alkyl” (or “C₁₋₄ alkyl”) means n-, iso-, sec- and t-butyl, n- and isopropyl, ethyl and methyl.

The term “C₀” as employed in expressions such as “C₀₋₆ alkyl” means a direct covalent bond.

The term “C₂-C₆ alkenyl” (or “C₂₋₆ alkenyl”) refers to a linear or branched chain alkenyl group having from 2 to 6 carbon atoms, and is selected from the hexyl alkenyl and pentyl alkenyl isomers, 1-, 2- and 3-butenyl, 1- and 2-isobutenyl, 1- and 2-propenyl, and ethenyl. “C₂-C₄ alkenyl” (or “C₂₋₄ alkenyl”) has an analogous definition.

The term “C₃-C₈ cycloalkyl” (or “C₃₋₈ cycloalkyl”) refers to a cyclic ring selected from cyclopropyl, cyclobutyl, cyclopentyl, cyclohexyl, cycloheptyl and cyclooctyl. “C₃-C₆ cycloalkyl” has an analogous meaning.

The term “halogen” (or “halo”) refers to fluorine, chlorine, bromine and iodine (alternatively, fluoro, chloro, bromo, and iodo).

The term “C₁-C₆ haloalkyl” (or “C₁₋₆ haloalkyl”) means a C₁ to C₆ linear or branched alkyl group as defined above with one or more halogen substituents. The term “C₁-C₄ haloalkyl” (or “C₁₋₄ haloalkyl”) has an analogous meaning.

The term “aryl” refers herein to phenyl, naphthyl, anthryl, or phenanthryl.

The term “heteroaryl” refers to a 5- or 6-membered heteroaromatic ring containing from 1 to 3 heteroatoms selected from N, O and S and a balance of carbon atoms. Representative examples of 5- or 6-membered heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

The term “carbocycle” (and variations thereof such as “carbocyclic” or “carbocyclyl”) as used herein refers to a C₃ to C₈ monocyclic, saturated or unsaturated ring or a C₇ to C₁₂ bicyclic ring system in which the rings are independent or fused and in which each ring is saturated or unsaturated. The carbocycle may be attached at any carbon atom which results in a stable compound. The fused bicyclic carbocycles are a subset of the carbocycles; i.e., the term “fused bicyclic carbocycle” generally refers to a C₇ to C₁₀ bicyclic ring system in which each ring is saturated or unsaturated and two adjacent carbon atoms are shared by each of the rings in the ring system. A subset of the fused bicyclic carbocycles are those bicyclic carbocycles in which one ring is a benzene ring and the other ring is saturated or unsaturated, with attachment via any carbon atom that results in a stable compound. Representative examples of this subset include the following:

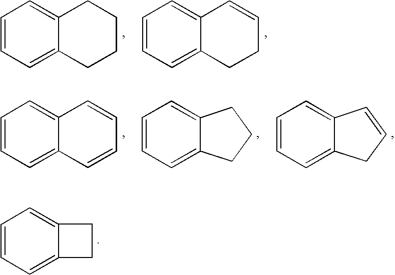

As used herein, the term “fused carbocyclic ring system” refers to a carbocycle as defined above which is fused to a phenyl ring. Representative examples include:

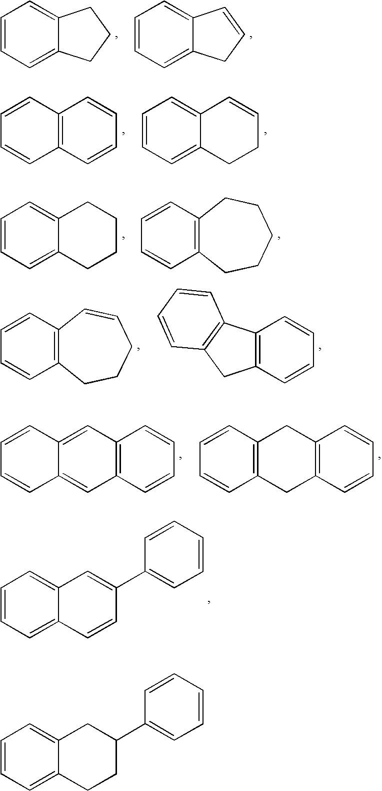

The term “substituted” (which appears in such expressions as “substituted with from 1 to 7 substituents”) includes mono- and poly-substitution by a named substituent to the extent such single and multiple substitution is chemically allowed and results in a chemically stable compound.

The symbol 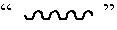
in front of an open bond in the structural formula of a group marks the point of attachment of the group to the rest of the molecule.

The term “catalytic amount” refers herein to any amount of a reagent which allows the reaction to proceed under less extreme conditions (e.g., at a lower reaction temperature) and/or in a shorter reaction time compared to the reaction conditions and/or reaction time in the absence of the reagent. A catalytic amount of a reagent is generally a substoichiometric amount of the reagent relative to the reactants, and herein is typically from about 0.001 to less than 1 molar equivalent (e.g., from about 0.001 to about 0.9 equivalent, or from about 0.01 to about 0.5 equivalent) per mole of reactant.

Combinations of substituents and/or variables are permitted only to the extent such combinations result in chemically stable compounds.

When any variable (e.g., Z¹, Z², R^{a}, and R^{b}) occurs more than one time in any constituent or in any formula, its definition on each occurrence is independent of its definition at very other occurrence.

Abbreviations used in the instant specification include the following:
AcOH=acetic acid
AIDS=acquired immunodeficiency syndrome
ARC=AIDS related complex
DEAD=diethylazodicarboxylate
DMEU=1,3-dimethyl-2-imidazolidinone (or N,N′-dimethylethyleneurea)
DMF=N,N-dimethylformamide
DMPU=1,3-dimethyl-3,4,5,6-tetrahydro-2(1H)-pyrimidinone (or N,N′-dimethylpropyleneurea)
DIPA=diisopropylamine
DMSO=dimethylsulfoxide
EDC=1-ethyl-3-(3-dimethylaminopropyl) carbodiimide
EDTA=ethylenediamine tetraacetic acid
EtOH=ethanol
g=gram(s)
GC =gas chromatography
h=hour(s)
HEDTA=hydroxyethylethylenediamine triacetic acid
HIV=human immunodeficiency virus
HPLC=high-performance liquid chromatography
Me=methyl
MeCN=acetonitrile
MeOH=methanol
min=minute(s)
Ms=mesyl (methanesulfonyl)
MTBE=methyl t-butyl ether
NBS=N-bromonsuccinimide
NMR=nuclear magnetic resonance
Ph=phenyl
n-PrOH=n-propyl alcohol
TEA=triethylamine
Tf=triflyl (trifluoromethanesulfonyl)
THF=tetrahydrofuran
Ts=tosyl

The following examples serve only to illustrate the invention and its practice. The examples are not to be construed as limitations on the scope or spirit of the invention.

### EXAMPLE 1

Preparation of 1,4-Butanesultam

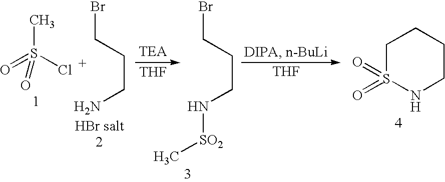

| | Weight | FW | Moles | Equiv. | Density | Volume |
|---|---|---|---|---|---|---|
| MsCl (1) | 2.36 Kg | 114.55 | 20.6 | 1.03 | 1.480 | 1.59 L |
| 3-bromo- | 4.40 Kg | 220 | 20.0 | 1.00 | | |
| propyl-amine | | | | | | |
| (2) HBr salt | | | | | | |
| TEA | 4.07 Kg | 101.19 | 40.2 | 2.01 | 0.726 | 5.60 L |
| THF | | | | | 43 + 4 + 8 = 55 L | |
| DIPA | 481 g | 101.19 | 4.75 | 0.25 | 0.722 | 666 mL |
| 1,10-Phenan- | 4.11 g | 180.21 | | | | |
| throline | | | | | | |
| n-BuLi, 1.6 M | | | | | | |
| in hexane | | | | | | |

The 3-bromopropylamine-HBr salt (2) and TBF (43 L) were placed in a 72 L round-bottomed-flask under N₂ and the resulting slurry was cooled to 0° C.

Two dropping funnels were fitted to the flask. One was charged with the TEA and the other with a solution of the MsCl (1) and THF (4 L). The contents of the addition funnels were added at roughly the same rate (the TEA was added slightly faster than the MsCl) while maintaining an internal reaction temperature below 10° C. The addition required 2 h. The resulting white suspension was warmed to 23° C. and aged for 1 h. The suspended solids (a mixture of TEA-HBr and TEA-HCl) were removed by filtration through a dry frit. The cake was washed with THF (8 L). The combined filtrate and cake-rinse, a THF solution of 3, was collected in a 100 L round-bottomed-flask under N₂. To the solution of 3 was added the 1,10-phenanthroline and the DIPA and the resulting solution was cooled to −30° C. The n-BuLi was added over about 4 h maintaining the internal temperature below −20° C. After 1.25 eq of the n-BuLi was added the reaction mixture became deep brown and the color remained as the addition was completed. The reaction mixture was warmed to 0° C. over 3 h. A small aliquot was removed, and partitioned between saturated NH₄Cl and EtOAc. The EtOAc was evaporated and the residue examined by ¹H NMR to confirm consumption of 3 and conversion to 4. To the reaction mixture at 0° C. was added saturated aqueous NH₄Cl (12 L, the first 1 L slowly, a heat kick to 6° C. was observed) and then brine (12 L). The phases were partitioned and the aqueous phase was extracted with EtOAc (20 L). The organic phases were combined, washed with brine (4 L) and then concentrated under vacuum to about 12 L. The solvent was switched to EtOAc (20 L used) maintaining a volume of 12 L. After the solvent switch, a yellow slurry resulted. n-Heptane (20 L) was added with stirring and the slurry was cooled to 5° C. After a 1 h age the solids were collected on a frit and rinsed with cold (5° C.) 3:5 EtOAc/n-heptane. The wet cake was dried for 24 h under a stream of dry N2 to provide 1.44 Kg (53% from 2) of sultam 4 as a crystalline yellow solid.

¹H NMR (CDCl₃, 400 mHz) δ 4.36 (br s, 1H), 3.45 (m, 2H), 3.10 (m, 2H), 2.24 (m, 2H), 1.64 (m, 2H).

### EXAMPLE 1B

Alternative Preparation of 1,4-Butanesultam

Step 1:

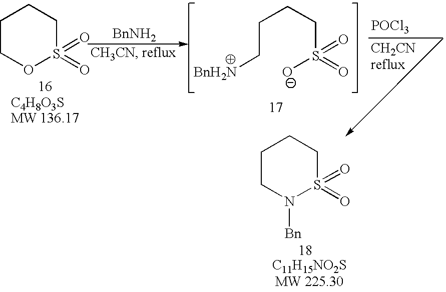

| Materials | MW | Amount | Moles | Equivalent |
|---|---|---|---|---|
| 1,4-Butane sultone | 136.17 | 68.10 g | 0.5000 | 1 |
| Benzylamine | 107.16 | 69.70 g | 0.6500 | 1.3 |
| Acetonitrile | | 625 mL | | |
| Phosphorus oxychloride | 153.33 | 153.33 g | 1.000 | 2 |

A solution of 1,4-butane sultone 16 (68.10 g, 0.5000 moles) and benzylamine (69.70 g, 0.6500 moles) in acetonitrile (625 mL) was refluxed at 82° C. for 24 hours, with the reaction monitored by ¹H NMR until conversion of 16 to 17 was >98%. While the resulting slurry was cooled to 50° C., phosphorus oxychloride (153.33 g, 1.000 moles) was slowly added via a dropping funnel. After complete addition, the mixture was refluxed at 82° C. for 8 hours, with the reaction monitored by HPLC until conversion was >98%. The reaction mixture was concentrated to remove acetonitrile, and the residue was cooled to 0-5° C. and neutralized with 20% sodium hydroxide to pH=7. The resulting mixture was extracted with IPAc (3×350 mL), and the combined extracts were washed with 10% sodium bicarbonate (2×100 mL) and 25% of brine (100 mL). The resulting clear solution was concentrated and solvent switched to methanol (total volume 1000 mL), which was used in the next step of the reaction. For compound 18: ¹H NMR (CDCl₃, 400 MHz) δ: 7.38-7.32 (m, 5H), 4.32 (s, 2H), 3.23 (m, 2H), 3.11 (m, 2), 2.22 (m, 2H), 1.62 (m, 2H).

Step 2:

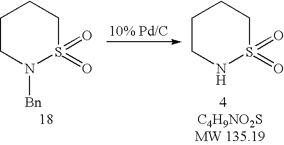

| Materials | MW | Amount | Moles | Equivalent |
|---|---|---|---|---|
| N-Benzyl-1,4-butanesultam | 225.30 | | 0.5000 | 1 |
| 10% Pd/C | | 12.0 g | | 10% wt |
| 1 N HCl (aqueous) | | 80 mL | | |
| Solka Flock | | 20 g | | |

To a solution of N-Benzyl-1,4-butanesultam 18 (0.5000 moles) in methanol (total volume 1000 mL) and 1 N HCl aqueous (80 nL) was added 10% Pd/C (12.0 g). The resulting slurry was submitted to hydrogenation at 40° C., 45 psi for 24 hours, with the reaction monitored by HPLC until conversion of 18 to 4 was >99%. The reaction mixture was cooled to ambient temperature and filtered by passing through a pad of Solka Flock (20 g) and washed with methanol (3×100 mL). The combined filtrates were concentrated to remove the methanol, and a crystalline solid was precipitated out during the concentration. To the slurry solution was added heptane/MTBE (3:2, 100 mL). The resulting mixture was cooled to 0° C., and aged for 0.5 hour. The crystalline solid was filtered off and washed with cold heptane/MTBE (3:2, 50 mL), and dried under vacuum with a nitrogen sweep to give 1,4-butanesultam 4 (49.8 g, 74% overall from 16).

### EXAMPLE 2

Preparation of 5-(1,1-dioxido-1,2-thiazinan-2-yl)-N-(4-fluorobenzyl)-8-hydroxy-1,6-naphthyridine-7-carboxamide from methyl 5-bromo-8-hydroxy-1,6-naphthyridine-7-carboxylate

Step 1: 5-Bromo-8-hydroxy-1,6-naphthyridine-7-carboxylic acid methyl ester

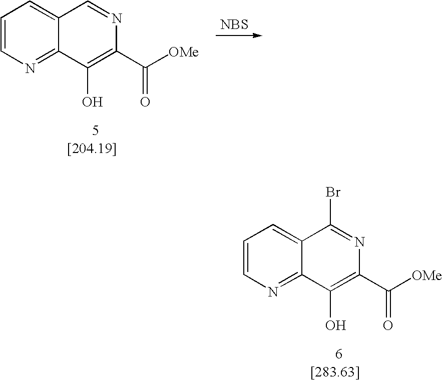

N-bromosuccinimide (7.83 g, 44.0 mmol) was added to a solution of 8-hydroxy-1,6-naphthyridine-7-carboxylic acid methyl ester (5, 8.17 g, 40.0 mmol) in chloroform (32 mL) over 20 min maintaining the temperature at 20-50° C. and the mixture was aged for 30 min at 50° C. The mixture became a thick, stirrable slurry and HPLC analysis indicated <2% starting material remaining. The mixture was cooled to 30° C. over 15 min. MeOH (64 mL) was added over 30 min then a 1:1 mixture of MeOH-water (64 nL) was added over 30 min. The mixture was cooled to −40° C. over 30 min and aged at −40° C. for 30 min. The cold mixture was filtered and the solid was washed with 1:1 MeOH:water (100 mL) at 10-20° C. The off white crystalline solid was dried under a stream of nitrogen to provide 10.48 g (93% yield) of 5-bromo-8-hydroxy-1,6-naphthyridine-7-carboxylic acid methyl ester (6)

HPLC retention times: 5=2.2 min, 6=6.0 min, HPLC conditions: 150×4.6 mm ACE 3 C18 column, isocratic elution with 30% MeCN in 0.025% aq H₃PO₄ at 1 mL/min, 25° C. with detection at 254 nm.

HPLC retention times: 5=1.8 min, 6=3.1 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 6 (CDCl₃, 100 MHz): 169.7, 156.3, 154.5, 143.9, 137.1, 132.4, 128.0, 126.1, 124.2, 53.4.
Step 2: 5-Bromo-8-(4-toluenesulfonyloxy)-1,6-naphthyridin-7-carboxylic acid methyl ester

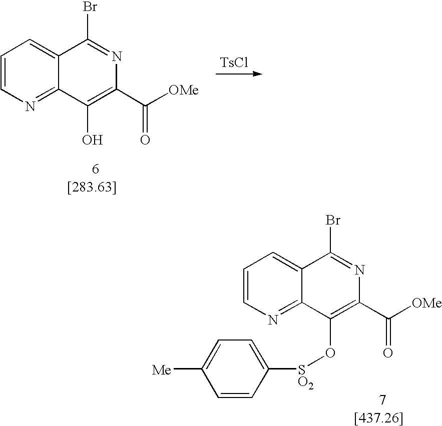

Triethylamine (0.759 g, 7.50 mmol) was added to a suspension of 5-bromo-8-hydroxy-1,6-naphthyridine-7-carboxylic acid methyl ester (6, 1.415 g, 5.000 mmol) in chloroform (5 mL) over 5 min maintaining the temperature at 20-50° C. to give a yellow suspension. p-Toluenesulfonyl chloride (1.15 g, 6.00 mmol) was added over 5 min maintaining the temperature at 20-40° C. to give a yellow solution. The mixture was aged at 40° C. for 2 h during which a crystalline solid precipitated out of the mixture and the color faded (HPLC analysis indicated <0.5% starting material remaining). The mixture was cooled to 20° C. over 15 min. MeOH (10 mL) was added over 30 min then a 1:1 mixture of MeOH:water (10 mL) was added over 30 min. The mixture was cooled to −40° C. over 30 min and aged at 40° C. for 30 min. The cold mixture was filtered and the solid was washed with 1:1 MeOH:water (10 mL), MeOH (5 mL), MTBE (10 mL) and hexanes (10 mL) all at 10-20° C. The off-white crystalline solid was dried under a stream of nitrogen to provide 2.112 g (97% yield) of 5-bromo-8-(p-toluenesulfonyloxy)-1,6-naphthyridine-7-carboxylic acid methyl ester (7)

HPLC retention times: 6=3.1 min, 7=12.4 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 7 (d6-DMSO, 100 MHz): 163.2, 157.0, 146.5, 145.8, 141.9, 141.3, 139.2, 137.2, 132.3, 130.4, 129.0, 127.6, 127.1, 53.3, 21.7.
Step 3: 5-(1,1-Dioxido-1,2-thiazinan-2-yl)-8-(4-toluenesulfonyloxy)-1,6-naphthyridine-7-carboxylic acid methyl ester.

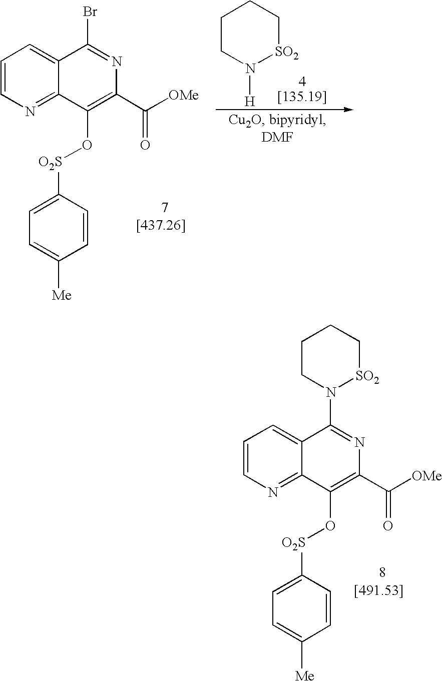

A mixture of 5-bromo-8-(p-toluenesulfonyloxy)-1,6-naphthyridine-7-carboxylic acid methyl ester (7, 2.186 g, 5.000 mmol), 1,4-butane sultam (4, 811 mg, 6.00 mmol), copper (I) oxide (858 mg, 6.00 mmol, <5 micron), 2,2′-bipyridyl (937 mg, 6.00 mmol) and DMP (10 mL) was degassed by stirring under a stream of nitrogen for 1 min and heated to 120° C. for 4 h. The brown suspension became a dark red solution with a small amount of undissolved copper (I) oxide remaining (HPLC: analysis indicated <0.5% starting material remaining). The mixture was diluted with chloroform (10 mL), Solkaflok (200 mg) was added and the resulting mixture was filtered through a plug of Solkaflok. The plug was washed with chloroform (10 mL) and the combined filtrates were stirred vigorously with a solution of EDTA disodium salt dihydrate (3.8 g, 10.2 mmol) in water (40 mL) while air was slowly bubbled in for 40 min. The upper aqueous phase became turquoise while the lower organic phase became yellow. The organic phase was washed with a solution of EDTA disodium salt (1.9 g, 5.1 mmol) in water (30 mL) and a solution of sodium bisulfate monohydrate (0.87 g, 6.3 mmol) in water (30 mL). Each of the above three aqueous phases was back extracted sequentially with one portion of chloroform (15 mL). The organic phases were dried over sodium sulfate and filtered. The dried organic extracts were concentrated and solvent switched to a final volume of 15 mL MeOH using a total of 30 mL MeOH for the switch at atmospheric pressure. Product crystallized during the solvent switch. The resulting slurry was cooled to 0° C. over 30 min and aged at 0° C. for 30 min. The slurry was filtered cold and the solid was washed with MeOH (15 mL). The off white solid was dried under a stream of nitrogen to provide 1.910 g (78%) of 5-(N-1,4-butanesultam)-8-(p-toluenesulfonyloxy)-1,6-naphthyridine-7-carboxylic acid methyl ester (8).
HPLC retention times: 7=12.4 min, 8=10.3 min, DMF=1.3 min, Bipy=1.5 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 8 (CDCl₃, 100 MHz): 164.2, 155.3, 151.9, 146.7, 145.4, 141.2, 137.8, 135.3, 133.6, 129.6, 128.9, 125.4, 124.3, 53.4, 52.9, 48.7, 24.2, 22.0, 21.7.

In an alternative procedure, HEDTA and THF have been used in the recovery of 8:

An HEDTA solution was prepared as follows: 37% aq HCl (710 g, 591 mL, 7.20 mol) was added to a slurry of 41.7 wt % aq Na₃HEDTA (4.952 kg, 3.869 L, 6.00 mol), and the temperature reached 35-40° C. Water (500 mL) was then added to the mixture and the mixture was cooled to 20-25° C. to give a homogeneous solution of 1.2M HEDTA (5.00 L, pH=7-8, d=1.23 g/mL). A total of 80 mL of this HEDTA solution was employed for the 20 mmol scale run described in the next paragraph.

A 200 mL flask was charged with the methyl ester 7(8.745 g, 20.0 mmol), sultam 4(2.974 g, 22.0 mmol), 2,2′-bipyridyl (3.436 g, 22 mmol), copper (I) oxide (3.148 g, 22.0 mmol) and DMF (20 mL) at 20-25° C. The mixture was degassed via three vacuum/nitrogen cycles and was then maintained under nitrogen atmosphere. The mixture was heated to 95-100° C. for 18 hours. The brown suspension became a deep red, nearly homogeneous mixture. The mixture was cooled to 25° C., and then TBF (20 mL) and Solkafloc (0.40 g) were added to the mixture. The mixture was filtered through a Solkafloc pad using THF (20 mL) to wash the pad. The HEDTA solution (40 mL) was added to the mixture. With agitation 5.25% sodium hypochlorite (bleach, 53 mL) was added over 20-40 min maintaining the temperature at 20-35° C., then TBF (20 mL) was added. The mixture was warmed to 45-50° C. and the phases were separated. The upper yellow organic phase was washed with HEDTA solution (20 mL) and the phases were separated at 45-50° C. Again, the upper yellow organic phase was washed with HEDTA solution (20 mL) and the phases were separated at 45-50° C. The upper yellow organic phase was washed with a solution containing 3 M aq sodium bisulfate (10 mL), saturated aq NaCl (15 mL) and water (10 mL) and the phases were separated at 45-50° C. Each of the lower aqueous phases obtained above were back extracted in the order they were obtained with a single portion of THF (20 mL) at 35-40° C. The main THF extract and the back extract were combined. The combined organic phases concentrated to about 40 mL and constant volume solvent switched to MeOH at atmospheric pressure, feeding in a total of 60 mL MeOH. The final residual volume was 40-50 mL and the final pot temperature was 60-65° C. The mixture was cooled to 10° C. over 30-60 minutes and aged at 5-10° C. for 30 minutes. The crystalline solid was filtered off on a filter pot and washed with MeOH (40 mL) at 20-25° C. The solid was dried under a stream of nitrogen to provide 8.116 g of product 8(83%).

The use of HEDTA and THF is beneficial because it avoids the use of less environmentally friendly solvents such as chloroform and dichloromethane. In addition, THF extractions are comparatively easier due to better phase cuts (less emulsion).
Step 4: 5-(1,1-Dioxido-1,2-thiazinan-2-yl)-8-hydroxy-1,6-naphthyridine-7-carboxylic acid methyl ester.

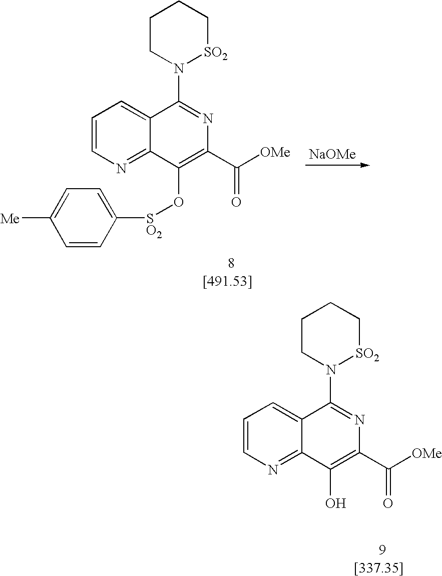

5-(N-1,4-butanesultam)-8-(p-toluenesulfonyloxy)-1,6-naphthyridine-7-carboxylic acid methyl ester (8,1.597 g, 3.250 mmol) was dissolved in DMF (3.25 mL) at 40° C. and transferred to a solution of 0.5M NaOMe in MeOH (16.25 mL, 8.125 mmol) over ca 1-2 min at 20-25° C. The resulting yellow homogenous mixture was heated to 50° C. and aged for 5 min (HPLC analysis indicated <0.5% starting material remaining). Mixture was cooled to 25° C. over 15 min and aged at 25° C. for 15 min during which a yellow crystalline precipitate was deposited. Acetic acid (390 mg, 6.50 mmol) was added over 1 min (yellow color faded) then water (32.5 mL) was added over 15 min at 25° C. The slurry was aged for 30 min 25° C. and filtered. The filter cake was washed with 1:1 MeOH:water (32.5 mL) and then with 1:1 MTBE:hexanes (8 mL). The filter cake was dried under a stream of nitrogen to provide 1.064 g (97%) of 5-(N-1,4-butanesultam)-8-hydroxy-1,6-naphthyridine-7-carboxylic acid methyl ester (2) as an off white crystalline solid.

HPLC retention times: 8=10.3 min, 9=2.9 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 9 (d6-DMSO, 100 MHz): 167.8, 154.4, 153.5, 143.9, 143.7, 135.2, 125.9, 125.2, 124.4, 53.2, 53.1, 49.1, 24.4, 21.9.
Step 5: 5-(1,1-Dioxido-1,2-thiazinan-2-yl)-N-(4-fluorobenzyl)-8-hydroxy-1,6-naphthyridine-7-carboxamide, monoethanolate.

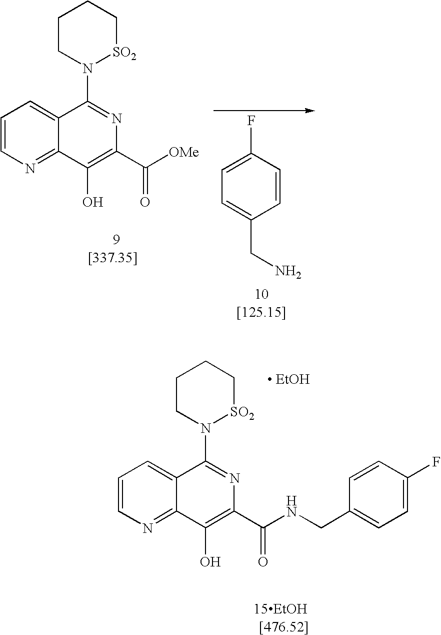

A suspension of 5-(N-1,4-butanesultam)-8-hydroxy-1,6-naphthyridine-7-carboxylic acid methyl ester (9, 1.012 g, 3.00 mmol) and 4-fluorobenzylamine (10, 1.314 g, 10.5 mmol) in EtOH (9.0 mL) was heated to 75-77° C. for 2 h during which the mixture became a yellow homogeneous solution (HPLC analysis indicated <0.5% starting material remaining). Acetic acid (0.630 mg, 10.5 mmol) was added over 1 min (yellow color faded) then water (9.0 mL) was added over 10 min at 75° C. An off white crystalline solid began to precipitate near the end of addition of the water. The slurry was cooled to 0° C. over 30 min then aged for 30 min at 0° C. and filtered. The filter cake was washed with 5% HOAc in 1:1 EtOH:water (5 mL) then with 1:1 EtOH:water (10 mL) and then with EtOH (5 mL). The filter cake was dried under a stream of nitrogen to provide 1.343 g (94%) of the monoethanolate of 5-(N-1,4-butanesultam)-N-(4-fluorobenzyl)-8-hydroxy-1,6-naphthyridine-7-carboxamide (15) as an off white crystalline solid.

HPLC retention times: 9=2.9 min, 15=6.7 min, 10=1.4 min, impurity present in 10=4.3 min, HPLC conditions: 150×4.6 mm ACE 3 C18 column, isocratic elution with 46% MeCN in 0.025% aq H₃PO₄ at 1 mL/min, 25° C. with detection at 254 nm;

HPLC retention time: 9=10.9 min, HPLC conditions: 150×4.6 mm ACE 3 C18 column, isocratic elution with 24% MeCN in 0.025% aq H₃PO₄ at 1 mL/min, 25° C. with detection at 254 nm.

¹H NMR (d6-DMSO, 400 MHz): 9.25 (t, J=6.4, 1H), 9.16 (d, J=8.4, 1H), 8.56 (d, J=8.4, 1H), 7.86 (dd, J=8.4, 1H), 7.41 (dd, J=8.4, 5.7, 2H), 7.16, t, J=8.8,2H), 4.60 (d, 6.3, 2H), 4.00-3.70 (m, 2H), 3.65-3.45 (m, 2H), 2.35-2.10 (m, 3H), 1.7 (m, 1H).
Step 6: Sodium salt of 5-(1,1-Dioxido-1,2-thiazinan-2-yl)-N-(4-fluorobenzyl)-8-hydroxy-1,6-naphthyridine-7-carboxamide

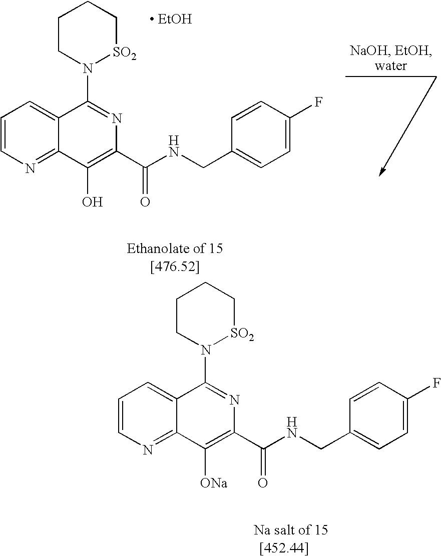

5-(N-1,4-Butanesultam)-N-(4-fluorobenzyl)-8-hydroxy-1,6-naphthyridine-7-carboxamide (15) monoethanolate (1.207 g, 2.533 mmol) was dissolved in a mixture of EtOH (24 mL) and water (11 mL) by heating to 78° C. for 1 h. A solution of 5M aq NaOH (0.608 mL, 3.04 mmol) was added over 15 min at 78° C. A yellow crystalline precipitate was deposited. The mixture was aged at 78° C. for 20 min, then cooled to 20° C. over 30 min and aged for 30 min at 20° C. The slurry was filtered and the filter cake was washed with 2:1 EtOH:water (5 mL) and EtOH (15 mL). The filter cake was dried under a stream of nitrogen to provide 1.088 g (95%) of 5-(N-1,4-butanesultam)-N-(4-fluorobenzyl)-8-hydroxy-1,6-naphthyridine-7-carboxamide sodium salt (15 sodium salt) as a yellow crystalline solid.

The Na salt was analyzed by differential scanning calorimetry at a heating rate of 10° C./min in an open cup under flowing nitrogen and was found to have a DSC curve exhibiting an endotherm with a peak temperature of about 348° C. and an associated heat of fusion of about 45 J/gm followed by an exotherm with a peak temperature of about 352° C. and an associated heat of fusion of about 45 J/gm.

The XRPD pattern of the Na salt was generated on a Philips Analytical X-ray powder diffraction (XRPD) instrument with XRG 3100 generator using a continuous scan from 2 to 40 degrees 2 theta over about 126 minutes. The resulting XRPD pattern was analyzed using Philips X'Pert Graphics and Identify software. Copper K-Alpha 1 radiation was used as the source. The experiment was run under ambient conditions. The XRPD pattern was found to have characteristic diffraction peaks corresponding to d-spacings of 12.63, 5.94, 5.05, 4.94, 4.81, 4.61, 4.54, 4.34, 3.88, 3.73, 3.49, 3.45, 3.22, 3.15, 3.12, and 2.86 angstroms.

Compound 15 is a potent HIV integrase inhibitor. The Na salt of Compound 15 exhibits superior oral absorption and improved pharmacokinetics in animal models compared to Compound 15 per se.

### EXAMPLE 3

Preparation of 5-(1,1-dioxido-1,2-thiazinan-2-yl)-N-(4-fluorobenzyl)-8-hydroxy-1,6-naphthyridine-7-carboxamide from N-(4-fluorobenzyl)-8-hydroxy-1,6-naphthyridine-7-carboxamide
Step 1: N-(4-Fluorobenzyl)-8-hydroxy-1,6-naphthyridine-7-carboxamide

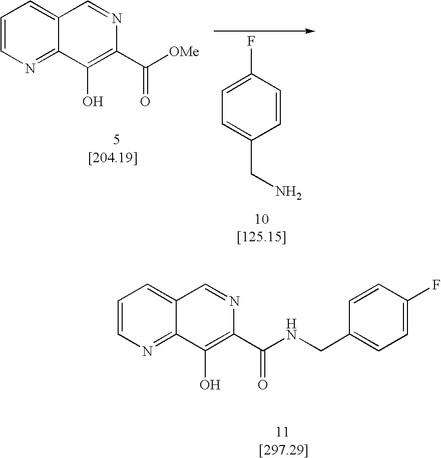

A mixture of 8-hydroxy-1,6-naphthyridine-7-carboxylic acid methyl ester (5, 408 mg, 2.00 mmol) and 4-fluorobenzylamine (10, 876 mg, 7.00 mmol) in EtOH (2 mL) were heated to 78° C. for 2 h giving homogeneous yellow solution (HPLC analysis indicated <0.5% starting material remaining). HOAc (420 mg, 7.00 mmol) was added (yellow color faded) and water (5.0 mL) was added over 10 min at 78° C. during which a white crystalline precipitate was deposited. The mixture was cooled to 20° C. over 10 min, aged for 40 min at 20° C. and filtered. The filter cake was washed with 1:1 MeOH:water (5 mL) and dried under a stream of nitrogen to provide 569 mg (96%) of N-(4-fluorobenzyl)-8-hydroxy-1,6-naphthyridine-7-carboxamide (11) as a white crystalline solid.

HPLC retention times: 5=1.8 min, 11=5.3 min. 10=1.4 min, impurity present in 10=4.3 min, HPLC conditions: 150×4.6 mm ACE 3 C18 column, isocratic elution with 46% MeCN in 0.025% aq H₃PO₄ at 1 mL/min, 25° C. with detection at 254 nm.
Step 2a: 5-Bromo-N-(4-fluorobenzyl)-8-hydroxy-1,6-naphthyridine-7-carboxamide via bromination of 11

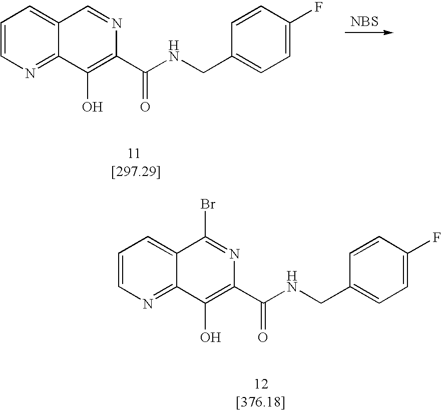

NBS (467 mg, 2.63 mmol) was added to a solution of N-(4-fluorobenzyl)-8-hydroxy-1,6-naphthyridine-7-carboxamide (11,743 mg, 2.50 mmol) in CHCl₃ (3 mL) over 15 min at 30° C. The mixture was aged 30 min at 30° C. (HPLC analysis indicated <0.5% starting material remaining). The mixture was constant volume (3 mL) solvent switched at atmospheric pressure to MeOH using a total of 6 mL MeOH. The mixture was cooled from 65° C. to 0° C. over 30 min and water (4 mL) was added over 10 min. The slurry was aged 30 min at 0° C. and filtered. The filter cake was washed with 1:1 MeOH/water (5 mL) and dried under a stream of nitrogen to provide 913 mg (97%) of 5-bromo-N-(4-fluorobenzyl)-8-hydroxy-1,6-naphthyridine-7-carboxamide (12) as a white crystalline solid.

HPLC retention times: 11=5.3 min, 12=13.0 min, HPLC conditions: 150×4.6 mm ACE 3 C18 column, isocratic elution with 46% MeCN in 0.025% aq H₃PO₄ at 1 mL/min, 25° C. with detection at 254 nm.
Step 2b: 5-Bromo-N-(4-fluorobenzyl)-8-hydroxy-1,6-naphthyridine-7-carboxamide via aminolysis of 6

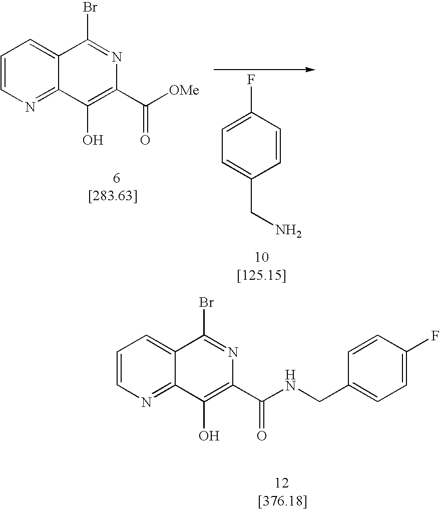

A mixture of 5-bromo-8-hydroxy-1,6-naphthyridine-7-carboxylic acid methyl ester (6, 567 mg, 2.00 mmol) and 4-fluorobenzylamine (876 mg, 7.00 mmol) in EtOH (2 mL) and DMF (0.5 mL) were heated to 78° C. for 1.5 h giving a yellow homogeneous solution (HPLC analysis indicated <0.5% starting material remaining). HOAc (420 mg, 7.00 mmol) was added (yellow color faded) and water (5.0 mL) was added over 10 min at 78° C. during which a white crystalline precipitate was deposited. The mixture was cooled to 20° C. over 10 min, aged for 40 min at 20° C. and filtered. The filter cake was washed with 1:1 MeOH:water (5 mL) and dried under a stream of nitrogen to provide 721 mg (96%) of N-(4-fluorobenzyl)-8-hydroxy-1,6-naphthyridine-7-carboxamide (12) as a white crystalline solid.

HPLC retention times: 6=3.1 min, 12=13.0 min, 10=1.4 min, impurity present in 7=4.3 min, HPLC conditions: 150×4.6 mm ACE 3 C18 column, isocratic elution with 46% MeCN in 0.025% aq H₃PO₄ at 1 mL/min, 25° C. with detection at 254 nm.
Step 3: 5-Bromo-N-(4-fluorobenzyl)-8-(4-toluenesulfonyloxy)-1,6-naphthyridine-7-carboxamide

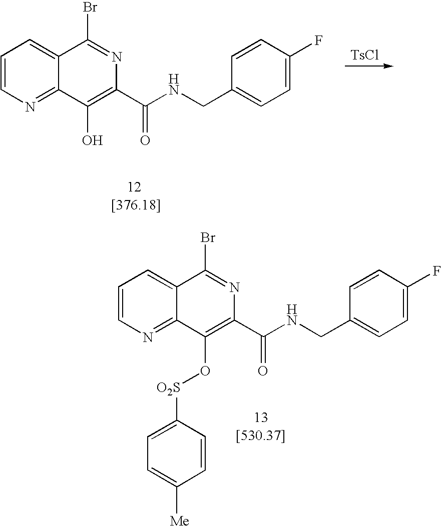

Triethylamine (12.75 g, 126.0 mmol) was added to a suspension of 5-bromo-N-(4-fluorobenzyl)-8-hydroxy-1,6-naphthyridine-7-carboxamide (12, 31.60 g, 84.00 mmol) in chloroform (84 mL) over 5 min maintaining the temperature at 22-27° C. to give a yellow cloudy solution. p-Toluenesulfonyl chloride (19.22 g, 100.8 mmol) was added over 5 min maintaining the temperature at 27° C. to give a yellow homogeneous solution (virtually no exotherm). The mixture was aged at 35-40° C. for 1 h (TALC analysis indicated <0.5% starting material remaining). The mixture was washed with 1M NaHSO₄ (126 mL) and then with water (126 mL) at 40° C. The resulting aqueous phases were each back extracted with one portion of chloroform (21 mL). The organic phases were dried over Na₂SO₄ (4 g) and filtered. The filtrates were concentrated to 84 mL residual volume at atmospheric pressure and constant volume (84 mL) solvent switched to MTBE using a total volume of 126 mL MIME at atmospheric pressure (a crystalline precipitate was deposited). MTBE (42 mL) was added to the mixture at 50° C. over 10 min. Hexanes (105 mL) were added to the mixture at 50° C. over 20 min. The mixture was cooled to 20° C. over 30 min and aged at 20° C. for 30 min. The mixture was filtered and the solid was washed with MTBE (105 mL) and hexanes (126 mL). The white crystalline solid was dried under a stream of nitrogen to provide 44.32 g (99% yield) of 5-bromo-N-(4-fluorobenzyl)-8-(p-toluenesulfonyloxy)-1,6-naphthyridine-7-carboxamide (13).

HPLC retention times: 12=13.0 min, 13=22.1 min, HPLC conditions: 150×4.6 mm ACE 3 C18 column, isocratic elution with 46% MeCN in 0.025% aq H₃PO₄ at 1 mL/min, 25° C. with detection at 254 nm.
Step 4: 5-(1,1-Dioxido-1,2-thiazinan-2-yl)-N-(4-fluorobenzyl)-8-(4-toluenesulfonyloxy)-1,6-naphthyridine-7-carboxamide

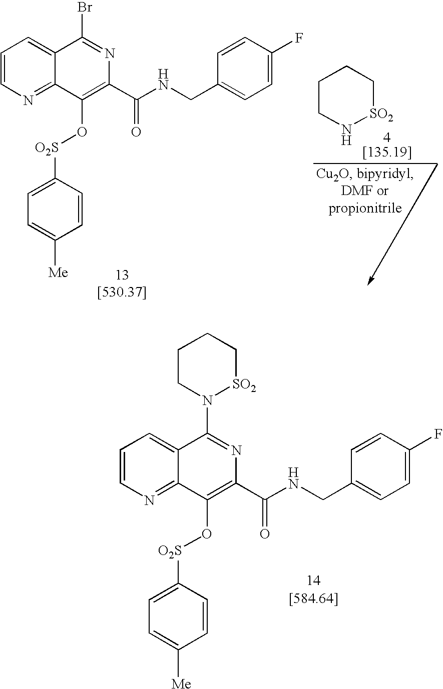

METHOD 1: A mixture of 5-bromo-N-(4-fluorobenzyl)-8-(p-toluenesulfonyloxy)-1,6-naphthyridine-7-carboxamide (13, 43.49 g, 82.00 mmol), 1,4-butane sultam (4, 13.30 g, 98.40 mmol), copper (I) oxide (14.08 g, 98.40 mmol, <5 micron), 2,2′-bipyridyl (15.57 g, 98.40 mmol) and DMF (164 mL) was degassed by stirring under a stream of nitrogen for 1 min and heated to 115-118° C. for 1 h. The brown suspension became a dark red solution with a small amount of undissolved copper (I) oxide remaining (HPLC analysis indicated <0.5% starting material remaining). The mixture was diluted with chloroform (164 mL), Solkaflok (4.1 g) was added and the resulting mixture was filtered through a plug of Solkaflok (4.1 g). The plug was washed with chloroform (164 mL) and the combined filtrates were stirred vigorously with a solution of 10% aq EDTA disodium salt dihydrate (500 mL, 0.13 mol) while 30% aq H₂O₂ (8.0 mL, 80 mmol) was added over 1 h. The upper aqueous phase became turquoise while the lower organic phase became yellow and the temperature rose from 24 to 32° C. A solution of 5M aq NaOH was added and the mixture was filtered through a pad of Solkaflok (2 g). The organic phase was stirred with 5% aq EDTA disodium salt dihydrate (400 mL, 5.4 mmol) and 30% aq H₂O₂ (1.0 mL, 10 mmol) The organic phase was washed with 5% aq EDTA disodium salt dihydrate (400 mL, 5.4 mmol) and a solution of sodium bisulfate monohydrate (16.3 g, 118 mmol) in water (326 ml). Each of the above four aqueous phases was back extracted sequentially with chloroform (2×41 mL). The combined organic phases were dried over sodium sulfate and filtered. The dried organic extracts were concentrated to a final volume of 100-150 mL and constant volume (100-150 mL) solvent switched at atmospheric pressure to a final volume of n-PrOH using a total of 4×50 mL n-PrOH for the switch. The final still head temperature was 95° C. and product crystallized near the end of the solvent switch. n-PrOH (200 mL) was added and the resulting slurry was aged at 90° C. for 10 min. The slurry was cooled to 20° C. over 30 min and aged at 20° C. for 1 h. The solid was filtered and washed with n-PrOH (100 mL), EtOH (100 mL) and MeOH (100 mL). The off white solid was dried under a stream of nitrogen to provide 33.22 g (69%) of 5-(N-0,1,4-butanesultam)-N-(4-fluorobenzyl)-8-(p-toluenesulfonyloxy)-1,6-naphthyridine-7-carboxamide (14) as a pale tan crystalline solid.

HPLC retention times: 13=22.1 min, 14=14.8 min, DMF=1.3 min, Bipy=1.5 min, HPLC conditions: 150×4.6 mm ACE 3 C18 column, isocratic elution with 46% MeCN in 0.025% aq H₃PO₄ at 1 mL/min, 25° C. with detection at 254 nm.

METHOD 2: A mixture of 5-bromo-N-(4-fluorobenzyl)-8-p-toluenesulfonyloxy)-1,6-naphthyridine-7-carboxamide (13, 4.932 g, 9.300 mmol), 1,4-butane sultam (4, 1.509 g, 11.16 mmol), copper (I) oxide (1.597 g, 11.16 mmol, <5 micron), 2,2′-bipyridyl (1.743 g, 11.16 mmol) and propionitrile (20 mL) was degassed by stirring under a stream of nitrogen for 1 min and heated to 95° C. for 5 h. The brown suspension became a dark red solution with a small amount of undissolved copper (I) oxide remaining (HPLC analysis indicated <0.5% starting material remaining). The mixture was diluted with chloroform (20 mL), Solkaflok (500 mg) was added and the resulting mixture was filtered through a plug of Solkaflok (1 g). The plug was washed with chloroform (20 mL) and the combined fitrates were stirred vigorously with a solution of EDTA disodium salt dihydrate (7.6 g, 20 mmol) in water (80 mL) while air was slowly bubbled in for 40 min. The upper aqueous phase became turquoise while the lower organic phase became yellow. The organic phase was washed with a solution of EDTA disodium salt dihydrate (3.8 g, 10 mmol) in water (60 mL) and a solution of sodium bisulfate monohydrate (1.7 g, 12 mmol) in water (30 mL). Each of the above three aqueous phases was back extracted sequentially with chloroform (2×20 mL). The organic phases were dried over sodium sulfate and filtered. The dried organic extracts were concentrated to a final volume of 30 mL and constant volume (30-35 mL) solvent switched at atmospheric pressure to n-PrOH using a total of 3×25 mL n-PrOH for the switch. The slurry was cooled to 0° C. over 2 h. The solid was filtered and washed with n-PrOH (10 mL) and MeOH (30 mL). The solid was dried under a stream of nitrogen to provide 4.137 g (76%) of 5-(N-1,4-butanesultam)-N-(4-fluorobenzyl)-8-(p-toluenesulfonyloxy)-1,6-naphthyridine-7-carboxamide (14) as a pale tan crystalline solid.
Step 5: 5-(1,1-Dioxido-1,2-thiazinan-2-yl)-N-(4-fluorobenzyl)-8-hydroxy-1,6-naphthyridine-7-carboxamide

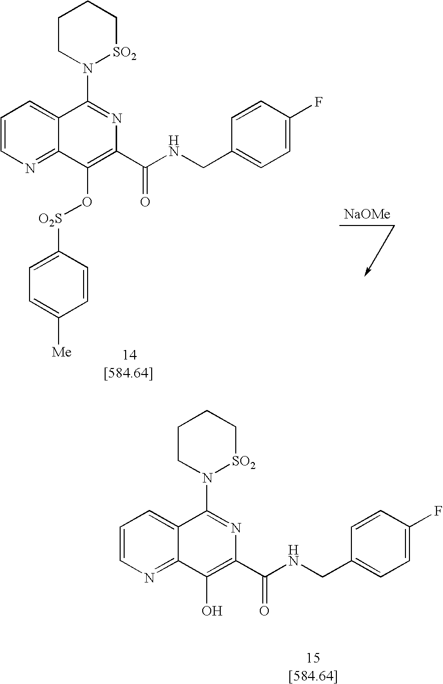

5-(N-1,4-butanesultam)-N-(4-fluorobenzyl)-8-(p-toluenesulfonyloxy)-1,6-naphthyridine-7-carboxamide (14, 3.800 g, 6.500 mmol) was dissolved in DMF (3.25 mL) at 40° C. and transferred to a solution of 0.5M NaOMe in MeOH (32.50 mL, 16.25 mmol) over ca 5 min at 20-25° C. DMF (0.50 mL) was used to complete the transfer. The resulting yellow homogenous mixture was heated to 50° C. (during which a yellow crystalline precipitate was deposited) and aged for 5 min (HPLC analysis indicated <0.5% starting material remaining). The mixture was cooled to 25° C. over 15 min and aged at 25° C. for 15 min. Acetic acid (781 mg, 13.0 mmol) was added over 1 min (yellow color faded) then water (65 mL) was added over 20 min at 25° C. The slurry was aged for 30 min 25° C. and filtered. The filter cake was washed with 1:1 MeOH:water (65 mL) and then with 1:1 MTBE:hexanes (18 mL). The filter cake was dried under a stream of nitrogen to provide 2.603 g (93%) of 5-(N-1,4-butanesultam)-N-(4-fluorobenzyl)-8-hydroxy-1,6-naphthyridine-7-carboxamide (8) as a pale tan crystalline solid.

HPLC retention times: 14=14.8 min, 15=6.7 min, HPLC conditions: 150×4.6 mm ACE 3 C18 column, isocratic elution with 46% MeCN in 0.025% aq H₃PO₄ at 1 mL/min, 25° C. with detection at 254 nm.

Step 6: Carbon treatment and recrystallization of 15

The crude free phenol 15 (23.00 g) from a detosylation of 14 was dissolved in a mixture of THF (230 mL), MeOH (13.8 mL) and MeOH (18.4 mL) at reflux (65° C.). Darco-G60 carbon (2.76 g) was added to the mixture and the mixture was aged at 65° C. for 30 min. The mixture was filtered through a 1 cm pad of Solkaflok on a 60 cc coarse sintered glass filter at 60-65° C. The pad was washed with a mixture of THF (46 mL), MeOH (2.3 mL) and MeOH (2.3 mL) at 60° C. The filtrates were constant volume (180-190 mL) solvent switched at atmospheric pressure to EtOAc using a total of 391 mL (still head temperature rose from 60° C. to 76° C.). n-Heptane (253 mL) was added to the mixture over 1 h while the temperature was allowed to fall from 77° C. to 45° C. The slurry was cooled to 5° C. over 1 h. The solid was filtered and washed with 2:1 n-heptane/EtOAc (92 mL) and n-heptane (69 mL). The solid was dried under a stream of nitrogen to provide 20.858 g (94% overall yield from 14) of 5-(N-1,4-butanesultam)-N-(4-fluorobenzyl)-8-hydroxy-1,6-naphthyridine-7-carboxamide (15) as an off white crystalline solid.

The following example illustrates the previous process for preparing Compound 15, and is provided for the purpose of comparison to Examples 1 and 2 above.

### EXAMPLE 4 (COMPARATIVE)

5-(1,1 dioxido-1,2-thiazinan-2-yl)-N-(4-fluorobenzyl)-8-hydroxy-1,6-naphthyridine-7-carboxamide (15)
Step 1: Preparation of 3-{[Methoxycarbonylmethyl-(toluene-4-sulfonyl)-amino]-methyl}-pyridine-2-carboxylic acid isopropyl ester

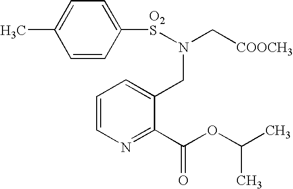

Isopropyl 3-(hydroxymethyl)pyridine-2-carboxylate, (200 g, 1.02 mol; prepared as in P. Ornstein et. al. J. Med, Chem. 1989, 32, 827), methyl N-[(4-methylphenyl)sulfonyl]glycinate (249 g, 1.02 mol), and triphenylphosphine (403 g, 1.5 mol) were dissolved in dry TBF (3000 mL) and cooled to 0° C. under nitrogen. The diethylazodicarboxylate (DEAD) (267.6 g, 1.5 mol) was dissolved in dry THF (250 mL) and placed in a 500 mL addition funnel. The DEAD was added dropwise over 1 hour. The ice bath was removed and the reaction was allowed to warm slowly to room temperature. After 2 hours, the reaction was checked by HPLC and some glycinate remained. More starting reagents were added and the reaction was left to stir at room temperature. After 30 min, the reaction was checked again and a very small amount of the glycinate remaining. The reaction mixture was concentrated down to a reddish-orange oil that was carried onto the next step.
Step 2: Preparation of methyl 8-hydroxy-1,6-naphthyridine-7-carboxylate

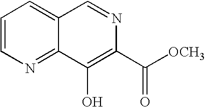

3-{[Methoxycarbonylmethyl-(toluene-4-sulfonyl)-amino]-methyl}-pyridine-2-carboxylic acid isopropyl ester (1.02 mol) was dissolved in dry methanol (4000 ml) and cooled to 0° C. under nitrogen. Then via addition funnel, sodium methoxide (137.8 g, 2.5 mol) was added slowly to avoid any exotherm. The reaction was stirred at zero degrees, and checked by HPLC after 1.5 hours and was found to be completed. The solvent was removed in vacuo to obtain a reddish-orange oil, which was partitioned between water (1 L) and ethyl acetate (1 L). The organic layer was back extracted with saturated sodium bicarbonate solution. The pH of the aqueous layer was adjusted to 7, and the layer was maintained at this pH while extracting with methylene chloride. The organic layer was dried with Na2SO₄, filtered, and the solvent was removed in vacuo to obtain a tan solid. The solid was dissolved in hot ethyl acetate, and the solution was filtered while hot to filter out any insoluble material. The product precipitated upon cooling. The precipitate was then filtered and dried in a vacuum oven. The filtrate was recrystallized by concentrating the filtrate and redissolving the resulting solid in a minimal amount of methylene chloride. Sufficient ethyl acetate was added to turn the solution slightly cloudy, after which the solution was boiled to reduce the volume, cooled, and the resulting crystals were filtered out and dried in a vacuum oven (152 g, 73%).

¹H NMR (CDCl₃, 500 MHz) δ 11.794 (5H,s), 9.2 (1H,dd, J=1.7 and 6.1 Hz), 8.8 (1H,s), 8.3 (1H,dd, J=1.5 and 9.7 Hz), 7.7 (1H, dd, J=4.2 and 12.4 Hz), 4.1 (3H,s) ppm.

ES MS exact mass calculated for C10H8N2O3 204.1869 (MH+), found 205.1.
Step 3: Preparation of methyl 5-bromo-8-hydroxy-1,6-naphthyridine-7-carboxylate

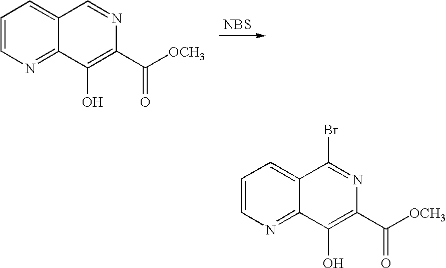

To a solution of methyl 8-hydroxy-1,6-naphthyridine-7-carboxylate from Step 2 (5.0 g, 24.49 mmol) in CH₂Cl₂ (100 mL) at room temperature was added N-bromosuccinimide (4.36 g, 24.49 mmol). The reaction was stirred for 1 hr. The solids were collected by filtration and dried in vacuo to afford the title compound as an off white solid (4.99 g, 72%).

FAB MS calcd for C₁₀H₇BrN₂O₂ 283 (MH⁺), found 283.

Step 4: 5-bromo-N-(4-fluorobenzyl)-8-hydroxy-1,6-naphthyridine-7-carboxamide

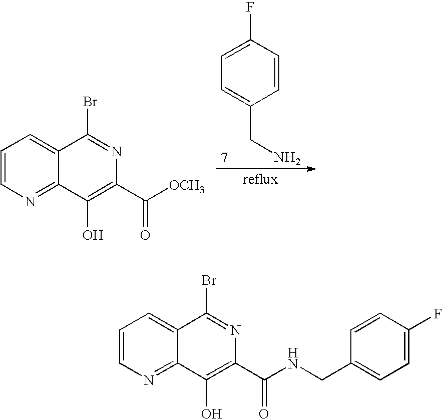

A slurry of the ester from Step 3 (0.50 g, 0.177 mmol) and 4-fluorobenzylamine (0.243 g, 1.94 mmol) in toluene (2 mL) were heated at reflux for 20 hrs. Upon cooling to room temperature, the resulting solids were collected by filtration and washed with methanol (3 mL) and then with diethyl ether (5 mL) to afford the title compound as a white solid (0.601 g, 90%).

¹H NMR (CDCl₃, 400 MHz) δ 9.20 (1H, d, J=4.3 Hz), 8.56 (1H, d, J=8.4 Hz), 8.17 (1H, m), 7.74 (1H, dd, J=8.4 and 4.3 Hz), 7.39 (2H, m), 7.07 (2H, t, J=8.6 Hz), 4.67 (2H, d, J=6.2 Hz) ppm.

FAB MS calcd for BrC₁₆H₁₁N₃O₂F 376 (MH⁺), found 376.
Step 5: 5-(1,1-dioxido-1,2-thiazinan-2-yl)-N-(4-fluorobenzyl)-8-hydroxy-1,6-naphthyridine-7-carboxamide (15) 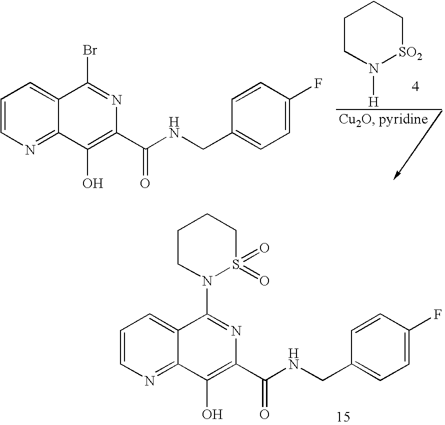
Run 1

To a mixture of 1,4 butanesultam (prepared as in White et al, J. Org Chem; 1987, 52: 2162) (1.00 g, 7.40 mmol), 5-bromo-N-(4-fluorobenzyl)-8-hydroxy-1,6-naphthyridine-7-carboxamide (3.06 g, 8.14 mmol), and Cu₂O (1.06 g, 7.40 mmol) under an atmosphere of argon was added pyridine (50 mL), and the suspension was stirred at reflux for 16 h. The reaction was allowed to cool to room temperature and filtered to remove the solids. The solids were washed with chloroform (500 mL). The resulting filtrate was evaporated to dryness and the residue was dissolved in chloroform (1 L) and vigorously stirred with a slurry of EDTA (4.0 g) in water (150 mL). After a period of 16 h, the chloroform extracts were dried (Na2SO₄) and evaporated in vacuo. The residue was purified by reverse phase HPLC. (Waters PrePak 500 cartridge C18, Gradient elution with Water:Acetonitrile 95:5 to 5:95 with 0.1% TFA at 75 mL/min over 45 mins). Lyophilization of the pure fractions afforded the title compound 15 as an off-white solid (0.660, 19%).

¹H NMR (d₆ DMSO, 400 MHz) δ 9.25 (1H, t, J=6.4 Hz), 9.16 (1H, d, J=4.1 Hz), 8.56 (1H, d, J=8.4 Hz), 7.86 (1H, dd, J=8.4 and 4.1 Hz), 7.41 (2H, dd, J=8.4 and 5.7 Hz), 7.16 (2H, t, J=8.8 Hz), 4.60 (2H, d, J=6.3 Hz) 4.00-3.70(2H, m), 3.65-3.45 (2H, m), 2.35-2.10(3H, m), 1.70 (1H, m) ppm.

FAB MS calcd for C20H19FN4O4S 431 (MH⁺), found 431.

Run 2

To a mixture of 1,4 butanesultam (7.187 g, 53.17 mmol), 5-bromo-N-(4-fluorobenzyl)-8-hydroxy-1,6-naphthyridine-7-carboxamide (20.00 g, 53.17 mmol), and Cu₂O (7.607 g, 53.17 mmol) under an atmosphere of argon was added pyridine (500 mL), and the suspension was stirred at reflux for 16 h. The reaction was allowed to cool to room temperature and filtered to remove the solids. The solids were washed with chloroform. The resulting filtrate was evaporated to dryness and the residue was dissolved in chloroform (600 mL) and vigorously stirred with a saturated aqueous solution of disodium EDTA (450 mL). After 0.5 hr, the chloroform extracts were separated and treated again with a saturated aqueous solution of disodium EDTA (350 mL). After 0.5 hr, the chloroform extracts were separated, dried (Na2SO₄) and evaporated in vacuo. The sticky residue (23.0 g) was dissolved in refluxing 95% aqueous ethanol (900 mL) and filtered hot to remove traces of solid. The filtrate was cooled and concentrated in vacuo until a solid began to precipitate. The solution was allowed to crystallize at room temperature for 16 hrs. The resulting solids were collected by filtration, washed with diethyl ether and air dried to afford the crude product (12.5 g). The solids (12.5 g) were recrystallized from 95% aqueous ethanol (500 nL) to afford the product 15 as its monoethanolate (10.76 g, 42%).

¹H NMR (d₆ DMSO, 400 MHz) δ 9.25 (1H, t, J=6.4 Hz), 9.16 (1H, d, J=4.1 Hz), 8.56 (1H, d, J=8.4 Hz), 7.86 (1H, dd, J=8.4 and 4.1 Hz), 7.41 (2H, dd, J=8.4 and 5.7 Hz), 7.16 (2H, t, J=8.8 Hz), 4.60 (2H, d, J=6.3 Hz) 4.00-3.70(2H, m), 3.65-3.45 (2H, m), 2.35-2.10(3H, m), 1.70 (1H, m) ppm.

FAB MS calcd for C20H19FN4O4S 431 (MH⁺), found 431.

While the foregoing specification teaches the principles of the present invention, with examples provided for the purpose of illustration, the practice of the invention encompasses all of the usual variations, adaptations and/or modifications that come within the scope of the following claims.

1. A process for preparing a compound of Formula (VIII): 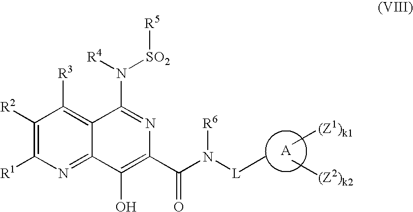
which comprises:
(C) reacting a compound of Formula (IIIa) or (IIIb): 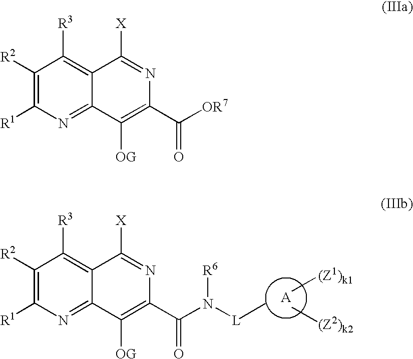
with a sulfonamide of Formula (IV):
R⁵SO₂—NHR⁴ (IV)
in solvent and in the presence of a copper promoter and a copper chelating agent to obtain a compound of Formula (Va) or (Vb): 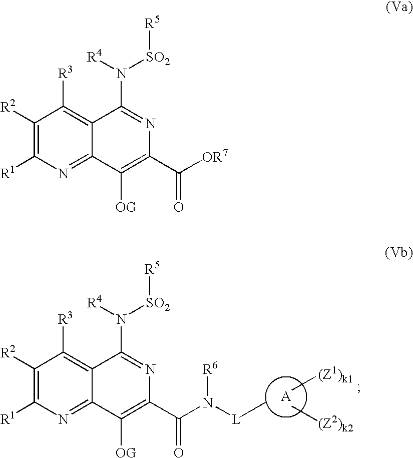
(D) when the compound resulting from Step C is Compound Va,
(D1) treating Compound Va with a phenol deprotecting agent to obtain a compound of Formula (VI): 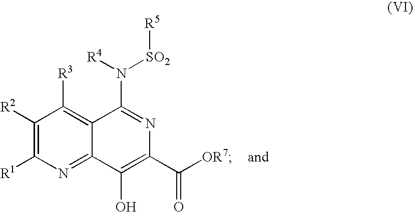
(D2) coupling Compound VI with an amine of Formula (VII): 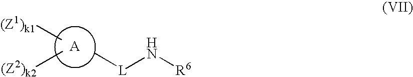
to obtain Compound VIII; and
(E) when the compound resulting from Step C is Compound Vb, treating Compound Vb with a phenol deprotecting agent to obtain Compound VIII;
wherein
A is phenyl or phenyl fused to a carbocycle to form a fused carbocyclic ring system;
G is a phenol protective group; or alternatively and with the proviso that the reactant in Step C is Compound IIIa, G and R⁷ together with the phenolic oxygen moiety and carbonyloxy moiety to which they are attached form a phenol protective cyclic group of formula: 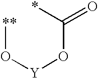
wherein * and ** respectively denote the points of fusion to ring carbons 7 and 8 in the naphthyridine ring; and Y is —C(R^{c})(R^{d})- or -B(R^{e})-;
L is a linker connecting a ring atom of A to the nitrogen of the —N(R⁶)-moiety, wherein L is
(i) a single bond connecting ring system A directly to N(R⁶),
(ii)-(C₁₋₆ alkyl)-,
(iii)-(C₂₋₆ alkenyl)-, or
(iv)-(C₀₋₆ alkyl)-(C₃₋₆ cycloalkyl)-(C₀₋₆ alkyl)-;
X is halo;
each Z¹ is a substituent on A independently selected from the group consisting of:
(1) —H,
(2) —C₁₋₆ alkyl, which is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂,
(3) —O—C₁₋₆ alkyl, which is optionally substituted with from 1 to 7 substituents each of which is independently halogen, —O—C₁₋₆ alkyl, —OH, or —SR^{a},
(4) —OH,
(5) halogen,
(6) —NO₂,
(7) —CN,
(8) —C(═O)R^{a},
(9)-CO₂R^{a},
(10) —SR^{a},
(11) —N(R^{b})₂,
(12) —C(═O)N(R^{a})₂,
(13) —SO₂R^{a},
(14) —N(R^{a})SO₂R^{a}, and
(15) —C₂₋₅ alkenyl;
k1 is an integer equal to zero, 1, 2, 3, 4 or 5;
each Z² is a substituent on A independently selected from the group consisting of:
(1)-H.
(2) aryl,
(3) —O-aryl,
(4)-C₁₋₆ alkyl-aryl,
(5) —O—C₁₋₆ alkyl-aryl,
(6) heteroaryl
(7)—O-heteroaryl,
(8) —C₁₋₆ alkyl-heteroaryl, and
(9) —O—C₁₋₆ alkyl-heteroaryl,
wherein the aryl in any of (2) to (5) or the heteroaryl in any of (6) to (9) is optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
k2 is an integer equal to zero, 1, or 2;
each of R¹, R² and R³ is independently:
(1) —H,
(2) —C₁₋₆ alkyl, which is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂,
(3) —O—C₁₋₆ alkyl, which is optionally substituted with from 1 to 7 substituents each of which is independently halogen, —O—C₁₋₆ alkyl, —OH, or —SR^{a},
(4) —OH,
(5) halogen,
(6) —NO₂,
(7) —CN,
(8) —C(═O)R^{a},
(9) —CO₂R^{a},
(10) —SR^{a},
(11) —N(R^{b})₂,
(12) —C(═O)N(R^{a})₂,
(13) —SO₂R^{a},
(14) —N(R^{a})SO₂R^{a}, and
(15) —C₂₋₅ alkenyl;
(16) aryl,
(17) —O-aryl,
(18) —C₁₋₆ alkyl-aryl,
(19) —O—C₁₋₆ alkyl-aryl,
(20) heteroaryl
(21)—O-heteroaryl,
(22) —C₁₋₆ alkyl-heteroaryl, and
(23)—O—C₁₋₆ alkyl-heteroaryl,
wherein the aryl in any of (16) to (19) or the heteroaryl in any of (20) to (23) is optionally substituted with from 1 to 5 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁴ is —H, —C₁₋₆ alkyl, or aryl; wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁵ is C₁₋₆ alkyl or aryl; wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
or alternatively R⁴ and R⁵ together with the —NSO₂— moiety to which they are attached form a sultam group of formula: 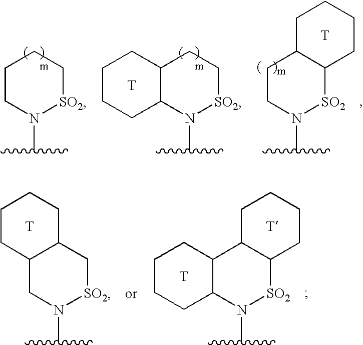
wherein T and T′ are each independently a 6-membered carbocyclic ring which is saturated, partially unsaturated, or aromatic; m is an integer equal to zero, 1, or 2; and the sultam group is optionally substituted with from 1 to 4 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁶ is —H or —C₁₋₆ alkyl, wherein the alkyl is optionally substituted with from 1 to 7 substituents independently selected from halogen, —O—C₁₋₆ alkyl, —O—C₁₋₆ haloalkyl, —N(R^{b})₂, and —CO₂R^{a};
R⁷ is —H, —C₁₋₆ alkyl or aryl, wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, —C(═O)N(R^{a})₂, or phenyl; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
each R^{a} is independently —H or —C₁₋₆ alkyl;
each R^{b} is independently —C₁₋₆ alkyl; and
R^{c} and R^{d} are each independently -H or —C₁₋₆ alkyl which is optionally substituted with from 1 to 7 substituents each of which is independently halogen, —O—C₁₋₆ alkyl, or —O-C₁₋₆ haloalkyl;
R^{e} is —C₁₋₆ alkyl, —O—C₁₋₆ alkyl, aryl, or —O-aryl; wherein the alkyl is optionally substituted with from 1 to 7 substituents each of which is independently halogen, —O—C₁₋₆ alkyl, or —O—C₁₋₆ haloalkyl; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —CHO, —C(═O)—C₁₋₆ alkyl, —CO2H, —CO₂—C₁₋₆ alkyl, —SH, —S—C₁₋₆ alkyl, —N(—C₁₋₆ alkyl)₂, —C(═O)NH₂, or —C(═O)N(—C₁₋₆ alkyl)₂;
and wherein each aryl is independently phenyl, naphthyl, anthryl, or phenanthryl;
and each heteroaryl is independently a 5- or 6-membered heteroaromatic ring containing from 1 to 3 heteroatoms selected from N, O and S and a balance of carbon atoms.

2. The process according to claim 1, wherein A is

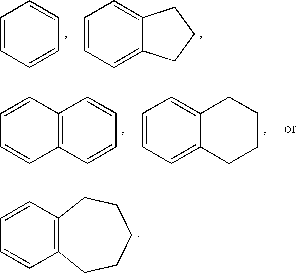

3. The process according to claim 2, wherein A is

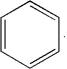

4. The process according to claim 1, wherein the —OG group on Compounds IIIa and Va or Compounds IIIb or Vb is an ether, a silyl ether, a carboxylic ester, a carbonate, a phosphinate or a sulfonate.

5. The process according to claim 4, wherein G is:
(1) —C₁₋₆ alkyl,
(2) —C₁₋₆ alkyl-aryl,
(3) —C₁₋₆ alkyl-O—C₁₋₆ alkyl,
(4) —C₁₋₆ alkyl-O—C₁₋₆ alkyl-aryl,
(5) —C₃₋₈ cycloalkyl,
(6) allyl,
(7)-Si(C₁₋₆ alkyl)ₙ(aryl)₃₋ₙ, wherein n is an integer equal to zero, 1, 2, or 3;
(8) —C(═O)—C₁₋₆ alkyl,
(9) —C(═O)-aryl,
(10) —C(═O)—C₁₋₆ alkyl-aryl,
(11) —C(═O)—O—C₁₋₆ alkyl,
(12) —C(═O)—O-aryl,
(13) —C(═O)—O—C₁₋₆ alkyl-aryl,
(14) —SO₂—C₁₋₆ alkyl,
(15) —SO₂—C₁₋₆ haloalkyl,
(16) —SO₂-aryl, or
(17) —P(O)(—C₁₋₆ alkyl)₂
wherein the aryl in (2), (4), (9), (10), (12), (13), or (16), and each aryl in (7) is optionally substituted with from 1 to 5 substituents each of which is independently halogen, -C₁₋₄ alkyl, —O—C₁₋₄ alkyl, or nitro.

6. The process according to claim 1, wherein L is —(C₁₋₆ alkyl)-.

7. The process according to claim 6, wherein L is —(CH₂)14-.

8. The process according to claim 1, wherein each Z¹ is independently selected from the group consisting of:
(1) —H,
(2)-C₁₋₄ alkyl, which is optionally substituted with from 1 to 5 substituents each of which is independently halogen, —O—C₁₋₄ alkyl, or —O—C₁₋₄ haloalkyl,
(3) —O-C₁₋₄ alkyl, which is optionally substituted with from 1 to 5 substituents each of which is independently halogen or —O-C₁₋₄ alkyl,
(4) halogen,
(5) —CN,
(6) —C(═O)R^{a},
(7) —CO₂R^{a},
(8) —SR^{a},
(9) —N(R^{b})₂,
(10) —C(═O)N(R^{a})₂,
(11) —SO₂R^{a},
(12) —N(R^{a})SO₂R^{a}, and
(13) —C₂₋₅ alkenyl; and
each Z² is independently selected from the group consisting of:
(1) —H,
(2) aryl,
(3) —O-aryl,
(4)-C₁₋₄ alkyl-aryl, and
(5)—O-C₁₋₄ alkyl-aryl,
wherein the aryl in any of (2) to (5) is phenyl or naphthyl and is optionally substituted with from 1 to 5 substituents each of which is independently halogen, -C₁₋₄ alkyl, -C₁₋₄ haloalkyl, —O—C₁₋₄ alkyl, or —O—C₁₋₄ haloalkyl.

9. The process according to claim 8, wherein k1 is an integer equal to zero, 1, 2 or 3; and k2 is an integer equal to zero or 1.

10. The process according to claim 1, wherein each of R¹, R² and R³ is independently:
(1) —H,
(2)-C₁₋₄ alkyl
(3)—(CH₂)₀₋₂CF₃,
(4) —O-C₁₋₄ alkyl,
(5) —O—(CH₂)₀₋₂CF₃,
(6) halogen selected from —F, —Cl and —Br,
(7) phenyl,
(8) —O-phenyl,
(9) —(CH₂)12-phenyl, or
(10) —O—(CH₂)12-phenyl,
wherein the phenyl in any of (7) to (10) is optionally substituted with from 1 to 4 substituents each of which is independently —F, —Cl, —Br, —C₁₋₄ alkyl, —C₁₋₄ haloalkyl, —O-C₁₋₄ alkyl, or —O-C₁₋₄ haloalkyl.

11. The process according to claim 10, wherein R¹ is —H, and R² and R³ are each as heretofore defined.

12. The process according to claim 11, wherein each of R¹, R² and R³ is —H.

13. The process according to claim 1, wherein R⁴ is —H, —C₁₋₄ alkyl, or phenyl, wherein the alkyl is optionally substituted with from 1 to 5 substituents each of which is independently halogen, —O-C₁₋₄ alkyl, or —O—C₁₋₄ haloalkyl; the phenyl is optionally substituted with from 1 to 4 substituents each of which is independently halogen, -C₁₋₄ alkyl, —C₁₋₄ haloalkyl, —O-C₁₋₄ alkyl, or —O-C₁₋₆ haloalkyl;
R⁵ is C₁₋₄ alkyl or phenyl, wherein the alkyl is optionally substituted with from 1 to 5 substituents each of which is independently halogen, —O-CA alkyl, or —O-C₁₋₄ haloalkyl; the phenyl is optionally substituted with from 1 to 4 substituents each of which is independently halogen, -C₁₋₄ alkyl, —C₁₋₄ haloalkyl, —O—C₁₋₄ alkyl, or —O—C₁₋₄ haloalkyl; or
alternatively R⁴ and R⁵ together with the —NSO₂— moiety to which they are attached form a sultam group of formula: 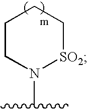
wherein m is an integer equal to zero, 1, or 2; and the sultam group is optionally substituted with from 1 to 3 substituents each of which is independently halogen, —C₁₋₄ alkyl, —C₁₋₄ haloalkyl, —O—C₁₋₄ alkyl, or —O—C₁₋₄ haloalkyl.

14. The process according to claim 13, wherein R⁴ and R⁵ together with the —NSO₂— moiety to which they are attached form a sultam group of formula: 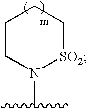
wherein m is an integer equal to zero, 1, or 2.

15. The process according to claim 1, wherein R⁶ is —H or —C₁₋₄ alkyl.

16. The process according to claim 21, wherein R⁶ is —H.

17. The process according to claim 1, wherein R⁷ is -C₁₋₄ alkyl.

18. The process according to claim 1, wherein the solvent in Step C is a polar aprotic solvent selected from the group consisting of nitrites, tertiary amides, ureas, ethers, N-alkylpyrrolidones, pyridines, halohydrocarbons, and esters.

19. The process according to claim 1, wherein Step C is conducted at a temperature in the range of from about 20 to about 300° C.

20. The process according to claim 1, wherein the copper promoter in Step C is copper metal, a copper oxide, or a copper salt selected from the group consisting of copper sulfides, halides, sulfonates, alkoxides, carboxylates, sulfates, thiocyanates, and nitrates.

21. The process according to claim 1, wherein the copper chelating agent in Step C is a polyamine, a polyaminocarboxylic acid, or a fused or singly bonded bipyridyl compound.

22. The process according to claim 1, wherein the copper promoter is employed in Step C in an amount in the range of from about 0.1 to about 10 equivalents per equivalent of Compound IIIa or IIIb.

23. The process according to claim 1, wherein the ratio of equivalents of copper chelating agent to copper promotor in Step C is in the range of from about 1:2 to about 2:1.

24. The process according to claim 1, wherein the sulfonamide IV is employed in Step C in the amount of from about 0.5 to about 5 equivalents per equivalent of Compound IIIa or IIIb.

25. The process according to claim 1, wherein the —OG group on Compound Va or Vb is an ether, a silyl ether, a carboxylic ester, or a sulfonate and treating in Step D1 or Step E comprises contacting Compound Va or Vb with an acid or base to cleave G to obtain Compound VI or VII.

26. The process according to claim 1, wherein the coupling of Step (D2) comprises reacting amine VII with Compound VI in solvent at a temperature in the range of from about 40 to about 200° C.

27. The process according to claim 1, which further comprises:
(B) treating a compound of Formula (IIa) or (IIb): 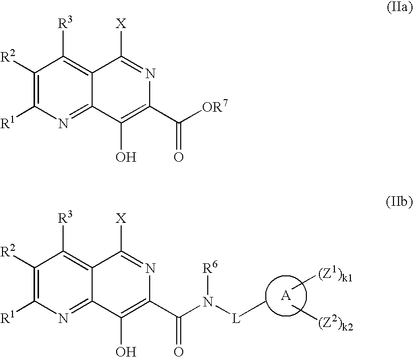
with a phenol protecting agent to obtain Compound (IIIa) or (IIIb).

28. The process according to claim 27, wherein the phenol protecting agent in Step B is selected from the group consisting of:
(i) a compound of formula G^{a}-Q, wherein Q is halide and G^{a} is:
(1) —C₁₋₆ alkyl,
(2) —C₁₋₆ alkyl-aryl,
(3) —C₁₋₆ alkyl-O-C₁₋₆ alkyl,
(4) —C₁₋₆ alkyl-O-C₁₋₆ alkyl-aryl,
(5) —C₃₋₈ cycloalkyl,
(6) allyl,
(7)-Si(C₁₋₆ alkyl)ₙ(aryl)₃₋ₙ, wherein n is an integer equal to zero, 1, 2, or 3;
(8) —C(═O)—C₁₋₆ alkyl,
(9) —C(═O)-aryl,
(10) —C(═O)—C₁₋₆ alkyl-aryl,
(11) —C(═O)—O—C₁₋₆ alkyl,
(12) —C(═O)—O-aryl,
(13) —C(═O)—O—C₁₋₆ alkyl-aryl,
(14) —SO₂—C₁₋₆ alkyl,
(15) —SO₂—C₁₋₆ haloalkyl, or
(16) —SO₂-aryl,
(ii) a sulfate compound of formula (G^{b})₂SO₄, wherein G^{b} is —C₁₋₆ alkyl or —C₁₋₆ alkyl-aryl; and
(iii) an anhydride of formula (G^{c} )20, wherein G^{c} is —C(═O)—C₁₋₆ alkyl, —C(═O)-aryl, or —C(═O)—C₁₋₆ alkyl-aryl;
(iv) a diazo compound of formula G^{d}-N2, wherein G^{d} is —C₁₋₆ alkylidenyl, —C₁₋₆ alkylidenyl-aryl or —C₃₋₈ cycloalkylidenyl;
wherein each aryl in (i), (iii), or (iv) is optionally substituted with from 1 to 5 substituents each of which is independently halogen, —C₁₋₄ alkyl, —O-C₁₋₄ alkyl, or nitro;
and wherein treatment with G^{a}-Q, (G^{b})₂SO₄, or (G^{c})₂O results in the attachment of G^{a}, G^{b}, or G^{c} as the phenol protective group G in Compound IIIa or IIIb, and treatment with G^{d}-N₂ results in the attachment of G^{d}H as the phenol protective group G in Compound IIIa or IIIb.

29. The process according to claim 27, which further comprises:
(A) contacting a compound of Formula (Ia) or (Ib): 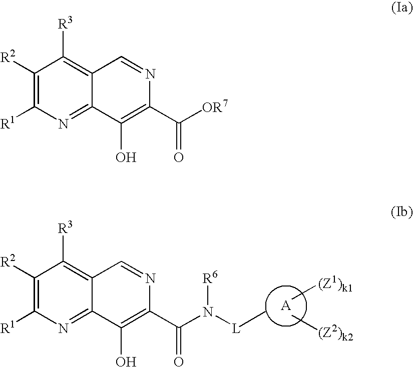
with a halogenating agent to obtain Compound (IIa) or (IIb).

30. The process according to claim 29, wherein the compound employed in Step A is Compound Ib, and the process further comprises coupling Compound (Ia) with an amine of Formula (VII): 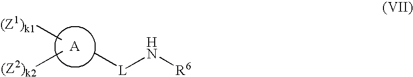
to obtain Compound IIb.

31. A process for preparing a compound of Formula (VIII′): 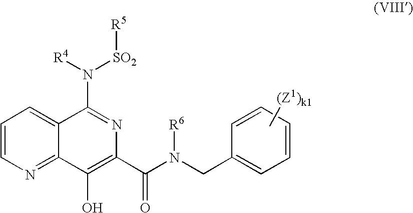
which comprises:
(C) reacting a compound of Formula (IIIa′) or (IIIb′): 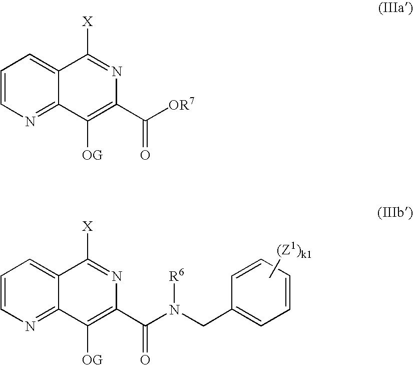
with a sulfonamide of Formula (IV):
R⁵SO₂—NHR⁴ (IV)
in solvent and in the presence of a copper promoter and a copper chelating agent to obtain a compound of Formula (Va′) or (Vb′): 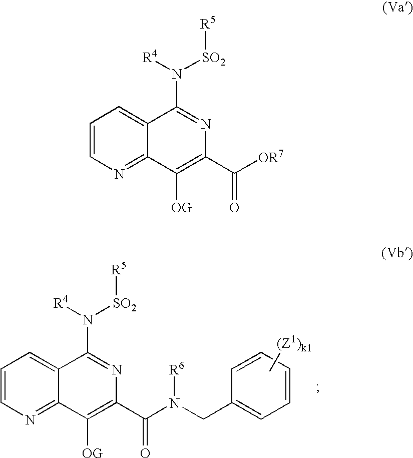
(D) when the compound resulting from Step C is Compound Va′,
(D1) treating Compound Va′ with a phenol deprotecting agent to obtain a compound of Formula (VI′): 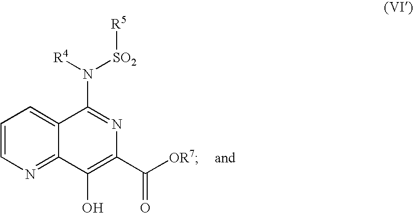
and
(D2) coupling Compound VI′ with an amine of Formula (VII′): 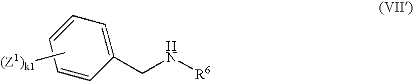
to obtain Compound VIII′; and
(E) when the compound resulting from Step C is Compound Vb′, treating Compound Vb′ with a phenol deprotecting agent to obtain Compound VIII′;
wherein:
each Z¹ is independently selected from the group consisting of:
(1)-H.
(2)—C₁₋₆ alkyl, which is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂,
(3) —O—C₁₋₆ alkyl, which is optionally substituted with from 1 to 7 substituents each of which is independently halogen, —O—C₁₋₆ alkyl, —OH, or —SR^{a},
(4) —OH,
(5) halogen,
(6) —NO₂,
(7) —CN,
(8) —C(═O)R^{a},
(9)-CO₂R^{a},
(10) —SR^{a},
(11) —N(R^{b})₂,
(12) —C(═O)N(R^{a})₂,
(13) —SO₂R^{a},
(14) —N(R^{a})SO₂R^{a}, and
(15) —C₂₋₅ alkenyl;
k1 is an integer equal to zero, 1, 2, 3, 4 or 5;
G is a phenol protective group; or alternatively and with the proviso that the reactant in Step C is Compound IIIa′, G and R⁷ together with the phenolic oxygen moiety and carbonyloxy moiety to which they are attached form a phenol protective cyclic group of formula: 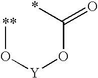
wherein * and ** respectively denote the points of fusion to ring carbons 7 and 8 in the naphthyridine ring; and Y is —C(R^{c})(R^{d})- or -B(R^{e})-;
X is halo;
R⁴ is —H, —C₁₋₆ alkyl, or aryl; wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁵ is C₁₋₆ alkyl or aryl; wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂; and the aryl is optionally substituted with from 1 to 7 substituents each of which is independently halogen, —C₁₋₆ alkyl, —C₁₋₆-haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
or alternatively R⁴ and R⁵ together with the —NSO₂— moiety to which they are attached form a sultam group of formula: 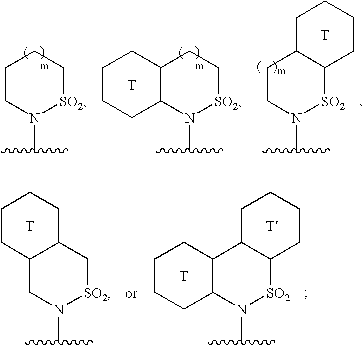
wherein T and T′ are each independently a 6-membered carbocyclic ring which is saturated, partially unsaturated, or aromatic; m is an integer equal to zero, 1, or 2; and the sultam group is optionally substituted with from 1 to 4 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
R⁶ is —H or —C₁₋₆ alkyl, wherein the alkyl is optionally substituted with from 1 to 7 substituents independently selected from halogen, —O—C₁₋₆ alkyl, —O—C₁₋₆ haloalkyl, —N(R^{b})₂, and —CO₂R^{a};
R⁷ is —H, —C₁₋₆ alkyl or aryl, wherein the alkyl is optionally substituted with from 1 to 7 substituents each of which is independently halogen, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, —C(═O)N(R^{a})₂, or phenyl; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —C(═O)R^{a}, —CO₂R^{a}, —SR^{a}, —N(R^{b})₂, or —C(═O)N(R^{a})₂;
each R^{a} is independently —H or —C₁₋₆ alkyl;
each R^{b} is independently —C₁₋₆ alkyl; and
R^{c} and R^{d} are each independently —H or —C₁₋₆ alkyl which is optionally substituted with from 1 to 7 substituents each of which is independently halogen, —O—C₁₋₆ alkyl, or —O—C′-6 haloalkyl;
R^{e} is —C₁₋₆ alkyl, —O—C₁₋₆ alkyl, aryl, or —O-aryl; wherein the alkyl is optionally substituted with from 1 to 7 substituents each of which is independently halogen, —O—C₁₋₆ alkyl, or —O—C₁₋₆ haloalkyl; and the aryl is optionally substituted with from 1 to 7 substituents each of which is independently halogen, —C₁₋₆ alkyl, —C₁₋₆ haloalkyl, —O—C₁₋₆ alkyl, —O—C₁₋₆ haloalkyl, —OH, —CN, —NO₂, —CHO, —C(═O)—C₁₋₆ alkyl, —CO2H, —CO2—C₁₋₆ alkyl, —SH, —S—C₁₋₆ alkyl, —N(—C₁₋₆ alkyl)₂, —C(═O)NH₂, or —C(═O)N(—C₁₋₆ alkyl)₂;
and wherein each aryl is independently phenyl, naphthyl, anthryl, or phenanthryl.

32. The process according to claim 31, which is a process for preparing Compound VIII′ which comprises:
(C) reacting a compound of Formula (IIIa′): 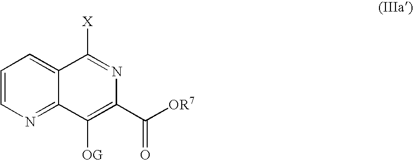
with a sulfonamide of Formula (IV):
R⁵SO₂—NHR⁴ (IV)
in solvent and in the presence of a copper promoter and a copper chelating agent to obtain a compound of Formula (Va′): 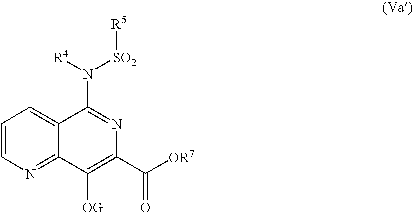
(D1) treating Compound Va′ with a phenol deprotecting agent to obtain a compound of Formula (VI′): 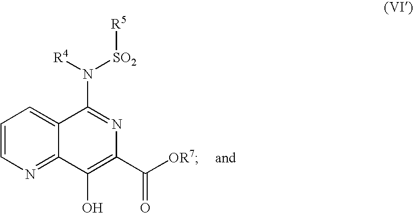
and
(D2) coupling Compound VI′ with an amine of Formula (VII′): 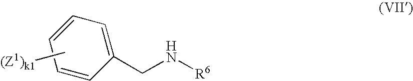
to obtain Compound VIII′.

33. The process according to claim 31, which is a process for preparing Compound VIII′, which comprises:
(C) reacting a compound of Formula (IIIb′): 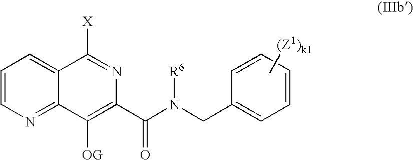
with a sulfonamide of Formula (IV):
R⁵SO₂—NHR⁴ (IC)
in solvent and in the presence of a copper promoter and a copper chelating agent to obtain a compound of Formula (Vb′): 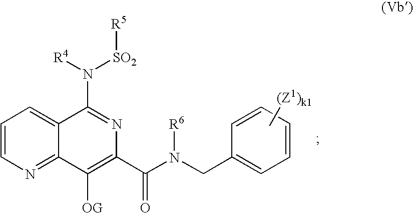
(E) treating Compound Vb′ with a phenol deprotecting agent to obtain Compound VIII′.

34. The process according to claim 31, wherein the —OG group on Compounds IIIa′ and Va′ or Compounds IIIb′ or Vb′ is an ether, a silyl ether, a carboxylic ester, a carbonate, a phosphinate or a sulfonate.

35. The process according to claim 34, wherein G is:
(1) —C₁₋₆ alkyl,
(2) —C₁₋₆ alkyl-aryl,
(3) —C₁₋₆ alkyl-O-C₁₋₆ alkyl,
(4) —C₁₋₆ alkyl-O-C₁₋₆ alkyl-aryl,
(5) —C₃₋₈ cycloalkyl,
(6) allyl,
(7)-Si(C₁₋₆ alkyl)ₙ(aryl)₃-n, wherein n is an integer equal to zero, 1, 2, or 3;
(8) —C(═O)—C₁₋₆ alkyl,
(9) —C(═O)-aryl,
(10) —C(═O)—C₁₋₆ alkyl-aryl,
(11) —C(═O)—O—C₁₋₆ alkyl,
(12) —C(═O)—O-aryl,
(13) —C(═O)—O—C₁₋₆ alkyl-aryl,
(14) —SO₂—C₁₋₆ alkyl,
(15) —SO₂—C₁₋₆ haloalkyl,
(16) —SO₂-aryl, or
(17) —P(O)(—C₁₋₆ alkyl)₂
wherein the aryl in (2), (4), (9), (10), (12), (13), or (16), and each aryl in (7) is optionally substituted with from 1 to 5 substituents each of which is independently halogen, —C₁₋₄ alkyl, —O—C₁₋₄ alkyl, or nitro.

36. The process according to claim 31, wherein each Z¹ is independently —H, —C₁₋₄ alkyl, —(CH₂)₀₋₂CF₃, —O-C₁₋₄ alkyl, —O—(CH₂)₀₋₂CF₃, or halo selected from —F, —Cl and —Br; and
k1 is zero, 1 or 2.

37. The process according to claim 31, wherein R⁴ is —H or —C₁₋₄ alkyl, and R⁵ is C₁₋₄ alkyl; or alternatively R⁴ and R⁵ together with the —NSO₂-moiety to which they are attached form a sultam group of formula: 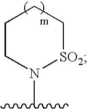
wherein m is an integer equal to zero, 1, or 2.

38. The process according to claim 31, wherein R⁷ is -C₁₋₄ alkyl.

39. The process according to claim 31, wherein:
the solvent in Step C is a polar aprotic solvent selected from the group consisting of nitriles, tertiary amides, ureas, ethers, N-alkylpyrrolidones, pyridines, halohydrocarbons, and esters;
the sulfonamide IV is employed in Step C in the amount of from about 0.8 to about 3 equivalents per equivalent of Compound IIIa′ or IIIb′; and
Step C is conducted at a temperature in the range of from about 70 to about 150° C.

40. The process according to claim 39, wherein
the copper promoter in Step C is copper metal, a copper oxide, or a copper salt selected from the group consisting of copper sulfides, halides, sulfonates, alkoxides, carbonates, carboxylates, sulfates, sulfites, thiocyanates, and nitrates;
the copper chelating agent in Step C is a polyamine, a polyaminocarboxylic acid, or a fused or singly bonded bipyridyl compound;
the copper promoter is employed in Step C in an amount in the range of from about 0.9 to about 3 equivalents per equivalent of Compound IIIa′ or IIIb′; and
the ratio of equivalents of copper chelating agent to copper promotor in Step C is in the range of from about 1:1.2 to about 1.2:1.

41. A process for preparing a compound of Formula (VIII″): 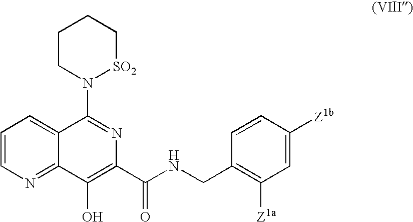
which comprises:
(C) reacting a compound of Formula (IIIa′): 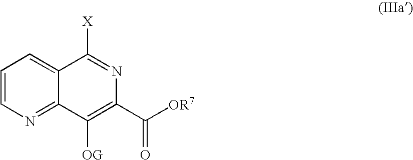
with sultam 4: 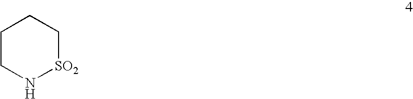
in solvent and in the presence of a copper promoter and a copper chelating agent to obtain a compound of Formula (Va″): 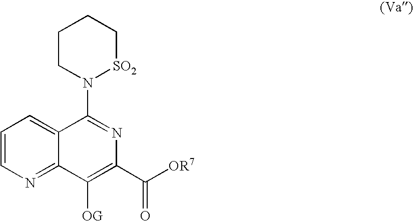
(D1) treating Compound Va″ with a phenol deprotecting agent to obtain a compound of Formula (VI″): 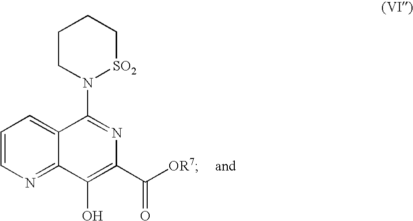
and
(D2) coupling Compound VI″ with an amine of Formula (VII″): 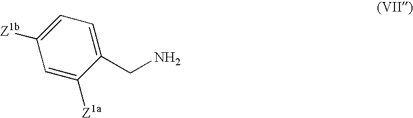
to obtain Compound VIII″; wherein:
G is a phenol protective group;
X is halo;
Z^{1a} and Z^{1b} are each independently —H or halo; and
R⁷ is —C₁₋₆ alkyl, phenyl, or benzyl.

42. The process according to claim 41, wherein the —OG group on Compound Va or Vb is an ether, a silyl ether, a carboxylic ester, or a sulfonate.

43. The process according to claim 42, wherein G is —SO₂—C₁₋₆ alkyl, —SO₂-C₁₋₆ haloalkyl, or —SO₂-aryl, wherein the aryl is optionally substituted with from 1 to 5 substituents each of which is independently halogen, —C₁₋₄ alkyl, —O—C₁₋₄ alkyl, or nitro.

44. The process according to claim 43, wherein G is CH₃SO₂—, CF₃SO₂—, or p-toluenesulfonyl.

45. The process according to claim 44, wherein G is p-toluenesulfonyl.

46. The process according to claim 41, wherein X is Cl or Br.

47. The process according to claim 41, wherein R⁷ is —C₁₋₄ alkyl.

48. The process according to claim 41, wherein one of Z^{1a} and Z^{1b} is fluoro or chloro, and the other of Z^{1a} and Z^{1b} is H, fluoro, or chloro.

49. The process according to claim 41, wherein amine VII″ in Step D2 is Compound 10: 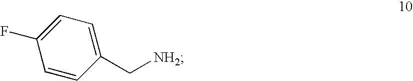
and Compound VIII″ is Compound 15:

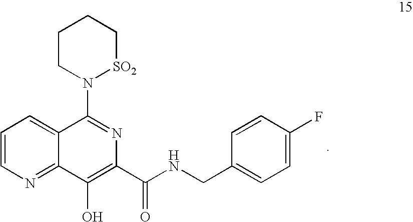

50. The process according to claim 41, wherein
the solvent in Step C is a polar aprotic solvent selected from the group consisting of nitrites, tertiary amides, ethers, N-alkylpyrrolidones, and pyridines;
Step C is conducted at a temperature in the range of from about 70 to about 150° C.;
the copper promoter in Step C is copper metal, a copper oxide, or a copper salt selected from the group consisting of copper sulfides, halides, sulfonates, alkoxides, carboxylates, sulfates, thiocyanates, and nitrates;
the copper chelating agent in Step C is a polyamine, a polyaminocarboxylic acid, or a fused or singly bonded bipyridyl compound;
the copper promoter is employed in Step C in an amount in the range of from about 0.9 to about 3 equivalents per equivalent of Compound IIIa′;
the ratio of equivalents of copper chelating agent to copper promotor in Step C is in the range of from about 1:1.2 to about 1.2:1; and
the sultam 4 is employed in Step C in the amount of from about 0.8 to about 3 equivalents per equivalent of Compound IIIa′.

51. A process for preparing compound of Formula (VIII″), 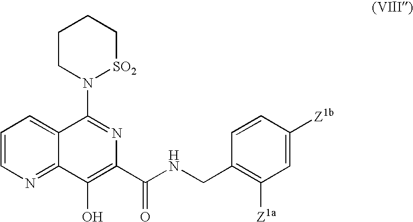
which comprises:
(C) reacting a compound of Formula (IIIb″): 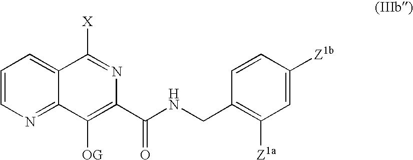
with sultam 4: 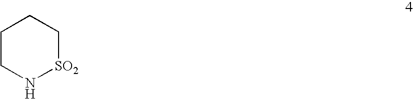
in solvent and in the presence of a copper promoter and a copper chelating agent to obtain a compound of Formula (Vb″): 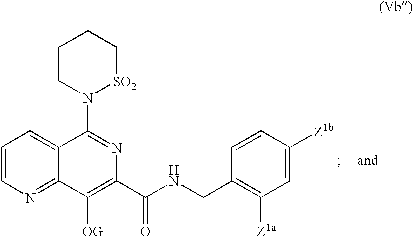
and
(E) treating Compound Vb″ with a phenol deprotecting agent to obtain Compound VIII″; wherein:
G is a phenol protective group;
X is halo; and
Z^{1a} and Z^{1b} are each independently —H or halo.

52. The process according to claim 51, wherein the —OG group on Compound Va or Vb is an ether, a silyl ether, a carboxylic ester, or a sulfonate.

53. The process according to claim 52, wherein G is —SO₂—C₁₋₆ alkyl, —SO₂—C₁₋₆ haloalkyl, or —SO₂-aryl, wherein the aryl is optionally substituted with from 1 to 5 substituents each of which is independently halogen, —C₁₋₄ alkyl, —O—C₁₋₄ alkyl, or nitro.

54. The process according to claim 53, wherein G is CH₃SO₂—, CF₃SO₂—, or p-toluenesulfonyl.

55. The process according to claim 54, wherein G is p-toluenesulfonyl.

56. The process according to claim 51, wherein X is Cl or Br.

57. The process according to claim 51, wherein one of Z^{1a} and Z^{1b} is fluoro or chloro, and the other of Z^{1a} and Z^{1b} is H, fluoro, or chloro.

58. The process according to claim 51, wherein Compound IIIb″ in Step C is: 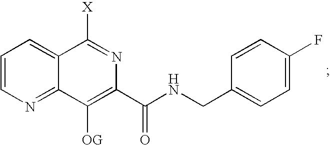
Compound Vb″ in Step E is 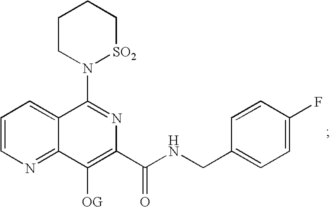
and Compound VIII″ is Compound 15

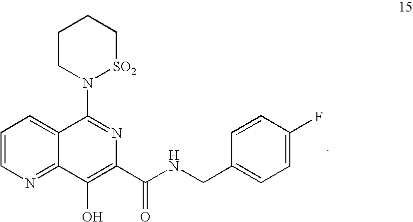

59. The process according to claim 51, wherein
the solvent in Step C is a polar aprotic solvent selected from the group consisting of nitrites, N,N-dialkyl amides, ureas, ethers, N-alkylpyrrolidones, and pyridines;
Step C is conducted at a temperature in the range of from about 70 to about 150° C.;
the copper promoter in Step C is copper metal, a copper oxide, or a copper salt selected from the group consisting of copper sulfides, halides, sulfonates, alkoxides, carboxylates, sulfates, thiocyanates, and nitrates;
the copper chelating agent in Step C is a polyamine, a polyaminocarboxylic acid, or a fused or singly bonded bipyridyl compound;
the copper promoter is employed in Step C in an amount in the range of from about 0.9 to about 3 equivalents per equivalent of Compound IIIb″,
the ratio of equivalents of copper chelating agent to copper promotor in Step C is in the range of from about 1:1.2 to about 1.2:1; and
the sultam 4 is employed in Step C in the amount of from about 0.8 to about 3 equivalents per equivalent of Compound IIIb″.

60. A process for preparing a compound of Formula (VIII″): 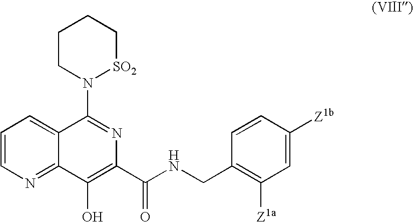
which comprises:
(C) reacting a compound of Formula (IIIa′): 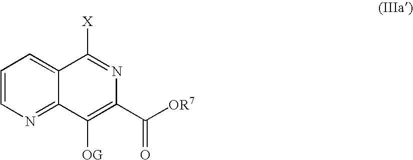
with sultam 4: 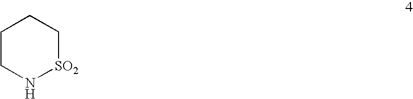
in solvent and in the presence of a catalytic amount of a copper (I) compound, a base and optionally a ligand to obtain a compound of Formula (Va″): 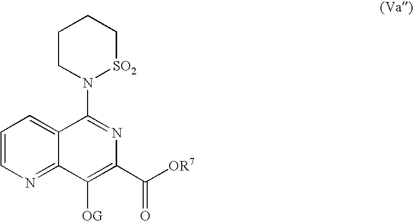
(D1) treating Compound Va″ with a phenol deprotecting agent to obtain a compound of Formula (VI): 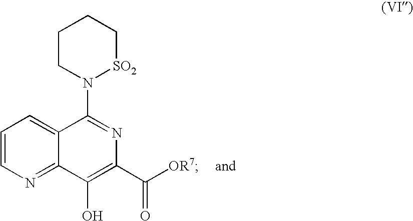
and
(D2) coupling Compound VI″ with an amine of Formula (VII″): 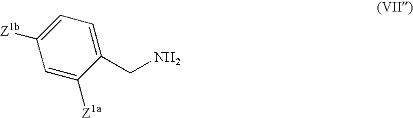
to obtain Compound VIII″; wherein G is a phenol protective group; X is halo; Z^{1a} and Z^{1b} are each independently —H or halo; and R⁷ is —C₁₋₆ alkyl, phenyl, or benzyl.
